(19) United States
(12) Patent Application Publication
(10) Pub. No.: US 2011/0081678 A1
(43) Pub. Date: Apr. 7, 2011

(54) METHOD FOR PRODUCING BETA-ALANYL-AMINO ACID OR DERIVATIVE THEREOF

(76) Inventors: Rie Takeshita, Kawasaki-shi (JP); Yasuaki Takakura, Kawasaki-shi (JP); Shunichi Suzuki, Kawasaki-shi (JP); Kenzo Yokozeki, Kawasaki-shi (JP)

(21) Appl. No.: 12/943,509

(22) Filed: Nov. 10, 2010

Related U.S. Application Data
(63) Continuation of application No. PCT/JP2009/058858, filed on May 12, 2009.

(30) Foreign Application Priority Data
May 12, 2008 (JP) 2008-125123

(51) Int. Cl. C12P 21/02; C07H 21/04; C12N 9/00; C12N 5/10

(52) U.S. Cl. 435/68.1; 536/23.2; 435/183; 435/325

(57) ABSTRACT

The present invention provides a method for efficiently producing a β-alanyl-amino acid (e.g., carnosine) or derivative thereof. Specifically, it provides a method for producing a β-alanyl-amino acid (e.g., β-alanyl-histidine) or derivative thereof, by reacting a β-alanyl ester or a β-alanyl amide and an amino acid or derivative thereof in the presence of an enzyme or an enzyme-containing product that has an ability to form the β-alanyl-amino acid (e.g., β-alanyl-histidine) or derivative thereof from the β-alanyl ester or the β-alanyl amide and the amino acid (e.g., histidine) or derivative thereof. The present invention also provides a protein which is able to catalyze production of a β-alanyl-amino acid or derivative thereof from a β-alanyl ester or a β-alanyl amide and an amino acid or derivative thereof, and a polynucleotide encoding said protein.

This application is a continuation under 35 U.S.C. §120 of PCT Patent Application No. PCT/JP2009/058858, filed May 12, 2009, which claims priority under 35 U.S.C. §119 to Japanese Patent Application No. 2008-125123, filed on May 12, 2008, which are incorporated in their entireties by reference. The Sequence Listing in electronic format filed herewith is also hereby incorporated by reference in its entirety (File Name: 2010-11-10T_US-450_Seq_List; File Size: 66 KB; Date Created: Nov. 10, 2010).

### BACKGROUND OF THE INVENTION

1. Field of the Invention

The present invention relates to a method for producing a β-alanyl-amino acid such as β-alanyl-histidine (carnosine) or derivatives thereof.

2. Brief Description of the Related Art

β-Alanyl-histidine, also called carnosine, is a β-alanyl-amino acid. β-Alanyl-histidine is a dipeptide made up of β-alanine and histidine, and is abundantly present in mammalian muscles, brain, and heart, including human. Although its roles in vivo have not been clearly elucidated, a pH adjusting action, an anti-inflammatory action, a tissue repairing action, an immunoregulatory action, an anti-oxidative action, an anti-protein glycosylation action, and the like, have all been previously reported.

A method for producing carnosine with an enzyme that catalyzes the formation of β-alanyl-histidine (i.e., carnosine) from β-alanine and histidine is known, β-Ala+His→β-Ala-His (see Skaper S D, Das S, Marshall F D. Some properties of a homocarnosine-carnosine synthetase isolated from rat brain. J Neurochem. 1973 December; 21(6): 1429-45; Horinishi H, Grillo M, Margolis F L. Purification and characterization of carnosine synthetase from mouse olfactory bulbs. J Neurochem. 1978 October; 31(4): 909-19; and US Patent Application Publication No. 2005/0287627). However, this enzyme requires ATP to catalyze the reaction.

It has also been reported that an imidazole-containing dipeptide such as carnosine can be synthesized by the above method using an imidazole dipeptide synthase derived from eel muscle (see Tsubone S, Yoshikawa N, Okada S, Abe H. Purification and characterization of a novel imidazole dipeptide synthase from the muscle of the Japanese eel Anguilla japonica. Comp Biochem Physiol B Biochem Mol Biol. 2007 April; 146(4): 560-7). This enzyme does not require ATP, but is very inefficient in its ability to produce carnosine.

### SUMMARY OF THE INVENTION

### Problem to be Solved by the Invention

In the light of the above prior art, it is an aspect of the present invention to provide a method for efficient production of a β-alanyl-amino acid such as carnosine or derivatives thereof.

### Means for Solving Problem

The reaction (β-AlaOMe+His→β-Ala-His) of producing or forming carnosine using a β-alanine ester and histidine as substrates was studied, and microorganisms were screened for an enzyme that catalyzes such a reaction. An enzymatic protein was purified from a microorganism thus identified, and its genetic information was further identified. The resulting enzymatic protein not only can catalyze the reaction of producing carnosine using a β-alanine ester and histidine as substrates, but also can generally catalyze reactions of producing a β-alanyl-amino acid or derivative thereof from a β-alanyl ester or a β-alanyl amide and an amino acid or derivative thereof.

Accordingly, the present invention provides the following:

It is an aspect of the present invention to provide a method for producing a β-alanyl-amino acid or derivative thereof, said method comprising reacting a substrate selected from the group consisting of a β-alanyl ester and a β-alanyl amide, with an amino acid or derivative thereof, in the presence of an enzyme or an enzyme-containing product, wherein said enzyme or enzyme-containing product is able to catalyze the formation of a β-alanyl-amino acid or derivative thereof from said substrate and the amino acid or derivative thereof.

It is a further aspect of the present invention to provide the method as described above, wherein the substrate is β-alanyl ester.

It is a further aspect of the present invention to provide the method as described above, wherein said amino acid or derivative thereof is an amino acid.

It is a further aspect of the present invention to provide the method as described above, wherein said β-alanyl-amino acid or derivative thereof is β-alanyl-histidine, said substrate is β-alanyl ester, and said amino acid or derivative thereof is histidine.

It is a further aspect of the present invention to provide the method as described above, wherein said enzyme-containing product is selected from the group consisting of A) a culture of a microorganism, B) a microbial cell separated from a culture of a microorganism, C) a treated microbial product of a culture of a microorganism, and D) combinations thereof.

It is a further aspect of the present invention to provide the method as described above, wherein said microorganism is of a genus selected from the group consisting of Rhodotorula, Tremella, Candida, Cryptococcus, Erythrobasidium, Sphingosinicella, and Aspergillus.

It is a further aspect of the present invention to provide the method as described above, wherein said microorganism is transformed with a gene encoding a protein selected from the group consisting of:

(A) a protein comprising the amino acid sequence of SEQ ID NO:3;

(B) a protein comprising the amino acid sequence of SEQ ID NO: 3, but wherein said sequence comprises one or more substitutions, deletions and/or insertions of one or several amino acids, and wherein said protein is able to catalyze the reaction of a substrate selected from the group consisting of a β-alanyl ester and a β-alanyl amide, with an amino acid or derivative thereof, to form a β-alanyl-amino acid or derivative thereof;

(C) a protein comprising the amino acid sequence of SEQ ID NO:5;

(D) a protein comprising the amino acid sequence of SEQ ID NO: 5, but wherein said sequence comprises one or more substitutions, deletions and/or insertions of one or several amino acids, and wherein said protein is able to catalyze the reaction of a substrate selected from the group consisting of a β-alanyl ester and a β-alanyl amide, with an amino acid or derivative thereof, to form a β-alanyl-amino acid or derivative thereof;

(E) a protein comprising the amino acid sequence of SEQ ID NO:7;

(F) a protein comprising the amino acid sequence of SEQ ID NO: 7, but wherein said sequence comprises one or more substitutions, deletions and/or insertions of one or several amino acids, and wherein said protein is able to catalyze the reaction of a substrate selected from the group consisting of a β-alanyl ester and a β-alanyl amide, with an amino acid or derivative thereof, to form a β-alanyl-amino acid or derivative thereof;

(G) a protein comprising the amino acid sequence of SEQ ID NO:21;

(H) a protein comprising the amino acid sequence of SEQ ID NO: 21, but wherein said sequence comprises one or more substitutions, deletions and/or insertions of one or several amino acids, and wherein said protein is able to catalyze the reaction of a substrate selected from the group consisting of a β-alanyl ester and a β-alanyl amide, with an amino acid or derivative thereof, to form a β-alanyl-amino acid or derivative thereof;

(I) a protein comprising the amino acid sequence of SEQ ID NO:25; and

(J) a protein comprising the amino acid sequence of SEQ ID NO: 25, but wherein said sequence comprises substitutions, deletions and/or insertions of one or several amino acids, and wherein said protein is able to catalyze the reaction of a substrate selected from the group consisting of a β-alanyl ester and a β-alanyl amide, with an amino acid or derivative thereof, to form a β-alanyl-amino acid or derivative thereof.

It is a further aspect of the present invention to provide the method as described above, wherein said β-alanyl-amino acid or derivative thereof is β-alanyl-histidine, said substrate is β-alanyl ester, and said amino acid or derivative thereof is histidine.

It is a further aspect of the present invention to provide the method as described above, wherein said microorganism is transformed with a polynucleotide selected from the group consisting of:

(a) a polynucleotide comprising the nucleotide sequence of the nucleotide numbers 40 to 1239 of SEQ ID NO:1;

(b) a first polynucleotide which hybridizes under stringent conditions with a second polynucleotide comprising a nucleotide sequence complementary to the nucleotide sequence of nucleotide numbers 40 to 1239 of SEQ ID NO:1, and wherein said first polynucleotide encodes a protein which is able to catalyze the reaction of a substrate selected from the group consisting of a β-alanyl ester and a β-alanyl amide, with an amino acid or derivative thereof, to form a β-alanyl-amino acid or derivative thereof;

(c) a polynucleotide comprising the nucleotide sequence of the nucleotide numbers 55 to 1239 of SEQ ID NO:1;

(d) a first polynucleotide which hybridizes under stringent conditions with a second polynucleotide comprising a nucleotide sequence complementary to the nucleotide sequence of nucleotide numbers 55 to 1239 of SEQ ID NO:1, and wherein said first polynucleotide encodes a protein which is able to catalyze the reaction of a substrate selected from the group consisting of a β-alanyl ester and a β-alanyl amide, with an amino acid or derivative thereof, to form a β-alanyl-amino acid or derivative thereof;

(e) a polynucleotide comprising the nucleotide sequence of the nucleotide numbers 91 to 1239 of SEQ ID NO:1;

(f) a first polynucleotide which hybridizes under stringent conditions with a second polynucleotide comprising a nucleotide sequence complementary to the nucleotide sequence of the nucleotide numbers 91 to 1239 of SEQ ID NO:1, and wherein said first polynucleotide encodes a protein which is able to catalyze the reaction of a substrate selected from the group consisting of a β-alanyl ester and a β-alanyl amide, with an amino acid or derivative thereof, to form a β-alanyl-amino acid or derivative thereof;

(g) a polynucleotide comprising the nucleotide sequence of SEQ ID NO:20;

(h) a first polynucleotide which hybridizes under stringent conditions with a second polynucleotide comprising a nucleotide sequence complementary to the nucleotide sequence of SEQ ID NO:20, and wherein said first polynucleotide encodes a protein which is able to catalyze the reaction of a substrate selected from the group consisting of a β-alanyl ester and a β-alanyl amide, with an amino acid or derivative thereof, to form a β-alanyl-amino acid or derivative thereof;

(i) a polynucleotide comprising the nucleotide sequence of SEQ ID NO:24;

(j) a first polynucleotide which hybridizes under stringent conditions with a second polynucleotide comprising a nucleotide sequence complementary to the nucleotide sequence of SEQ ID NO:24, and wherein said first polynucleotide encodes a protein which is able to catalyze the reaction of a substrate selected from the group consisting of a β-alanyl ester and a β-alanyl amide, with an amino acid or derivative thereof, to form a β-alanyl-amino acid or derivative thereof;

(k) a polynucleotide comprising the nucleotide sequence of SEQ ID NO:32; and

(l) a first polynucleotide which hybridizes under stringent conditions with a second polynucleotide comprising a nucleotide sequence complementary to the nucleotide sequence of SEQ ID NO:32, and wherein said first polynucleotide encodes a protein which is able to catalyze the reaction of a substrate selected from the group consisting of a β-alanyl ester and a β-alanyl amide, with an amino acid or derivative thereof, to form a β-alanyl-amino acid or derivative thereof.

It is a further aspect of the present invention to provide the method as described above, wherein said enzyme is selected from the group consisting of:

(A) a protein comprising the amino acid sequence of SEQ ID NO:3;

(B) a protein comprising the amino acid sequence of SEQ ID NO: 3, but wherein said sequence comprises one or more substitutions, deletions and/or insertions of one or several amino acids, and wherein said protein is able to catalyze the reaction of a substrate selected from the group consisting of a β-alanyl ester and a β-alanyl amide, with an amino acid or derivative thereof, to form a β-alanyl-amino acid or derivative thereof;

(C) a protein comprising the amino acid sequence of SEQ ID NO:5;

(D) a protein comprising the amino acid sequence of SEQ ID NO: 5, but wherein said sequence comprises one or more substitutions, deletions and/or insertions of one or several amino acids, and wherein said protein is able to catalyze the reaction of a substrate selected from the group consisting of a β-alanyl ester and a β-alanyl amide, with an amino acid or derivative thereof, to form a β-alanyl-amino acid or derivative thereof;

(E) a protein comprising the amino acid sequence of SEQ ID NO:7;

(F) a protein comprising the amino acid sequence of SEQ ID NO: 7, but wherein said sequence comprises one or more substitutions, deletions and/or insertions of one or several amino acids, and wherein said protein is able to catalyze the reaction of a substrate selected from the group consisting of a β-alanyl ester and a β-alanyl amide, with an amino acid or derivative thereof, to form a β-alanyl-amino acid or derivative thereof;

(G) a protein comprising the amino acid sequence of SEQ ID NO:21;

(H) a protein comprising the amino acid sequence of SEQ ID NO: 21, but wherein said sequence comprises one or more substitutions, deletions and/or insertions of one or several amino acids, and wherein said protein is able to catalyze the reaction of a substrate selected from the group consisting of a β-alanyl ester and a β-alanyl amide, with an amino acid or derivative thereof, to form a β-alanyl-amino acid or derivative thereof;

(I) a protein comprising the amino acid sequence of SEQ ID NO:25;

(J) a protein comprising the amino acid sequence of SEQ ID NO: 25, but wherein said sequence comprises one or more substitutions, deletions and/or insertions of one or several amino acids, and wherein said protein is able to catalyze the reaction of a substrate selected from the group consisting of a β-alanyl ester and a β-alanyl amide, with an amino acid or derivative thereof, to form a β-alanyl-amino acid or derivative thereof; and

(K) combinations thereof.

It is a further aspect of the present invention to provide a protein derived from a microorganism belonging to a genus selected from the group consisting of Rhodotorula, Tremella, Candida, Cryptococcus, and Erythrobasidium, and wherein said protein is able to catalyze the reaction of a substrate selected from the group consisting of a β-alanyl ester and a β-alanyl amide, with an amino acid or derivative thereof, to form a β-alanyl-amino acid or derivative thereof.

It is a further aspect of the present invention to provide a protein selected from the group consisting of:

(A) a protein comprising the amino acid sequence of SEQ ID NO:3;

(B) a protein comprising the amino acid sequence of SEQ ID NO: 3, but wherein said sequence comprises one or more substitutions, deletions and/or insertions of one or several amino acids, and wherein said protein is able to catalyze the reaction of a substrate selected from the group consisting of a β-alanyl ester and a β-alanyl amide, with an amino acid or derivative thereof, to form a β-alanyl-amino acid or derivative thereof;

(C) a protein comprising the amino acid sequence of SEQ ID NO:5;

(D) a protein comprising the amino acid sequence of SEQ ID NO: 5, but wherein said sequence comprises one or more substitutions, deletions and/or insertions of one or several amino acids, and wherein said protein is able to catalyze the reaction of a substrate selected from the group consisting of a β-alanyl ester and a β-alanyl amide, with an amino acid or derivative thereof, to form a β-alanyl-amino acid or derivative thereof;

(E) a protein comprising the amino acid sequence of SEQ ID NO:7; and

(F) a protein comprising the amino acid sequence of SEQ ID NO: 7, but wherein said sequence comprises one or more substitutions, deletions and/or insertions of one or several amino acids and wherein said protein is able to catalyze the reaction of a substrate selected from the group consisting of a β-alanyl ester and a β-alanyl amide, with an amino acid or derivative thereof, to form a β-alanyl-amino acid or derivative thereof;

It is a further aspect of the present invention to provide the protein as described above, wherein said β-alanyl-amino acid or derivative thereof is β-alanyl-histidine, said substrate is β-alanyl ester, and said amino acid or derivative thereof is histidine.

It is a further aspect of the present invention to provide a polynucleotide encoding the protein as described above.

It is a further aspect of the present invention to provide a polynucleotide selected from the group consisting of:

(a) a polynucleotide comprising the nucleotide sequence of nucleotide numbers 40 to 1239 of SEQ ID NO:1;

(b) a first polynucleotide which hybridizes under stringent conditions with a second polynucleotide comprising a nucleotide sequence complementary to the nucleotide sequence of nucleotide numbers 40 to 1239 of SEQ ID NO:1, and wherein said first polynucleotide encodes a protein which is able to catalyze the reaction of a substrate selected from the group consisting of a β-alanyl ester and a β-alanyl amide, with an amino acid or derivative thereof, to form a β-alanyl-amino acid or derivative thereof;

(c) a polynucleotide comprising the nucleotide sequence of nucleotide numbers 55 to 1239 of SEQ ID NO:1;

(d) a first polynucleotide which hybridizes under stringent condition with a second polynucleotide comprising a nucleotide sequence complementary to the nucleotide sequence of nucleotide numbers 55 to 1239 of SEQ ID NO:1, and wherein said first polynucleotide encodes a protein which is able to catalyze the reaction of a substrate selected from the group consisting of a β-alanyl ester and a β-alanyl amide, with an amino acid or derivative thereof to form a β-alanyl-amino acid or derivative thereof;

(e) a polynucleotide having the nucleotide sequence of nucleotide numbers 91 to 1239 of SEQ ID NO:1;

(f) a first polynucleotide which hybridizes under stringent conditions with a second polynucleotide comprising a nucleotide sequence complementary to the nucleotide sequence of the nucleotide numbers 91 to 1239 of SEQ ID NO:1, and wherein said first polynucleotide encodes a protein which is able to catalyze the reaction of a substrate selected from the group consisting of a β-alanyl ester and a β-alanyl amide, with an amino acid or derivative thereof, to form a β-alanyl-amino acid or derivative thereof;

(g) a polynucleotide comprising the nucleotide sequence of SEQ ID NO:32; and

(h) a first polynucleotide which hybridizes under stringent conditions with a second polynucleotide comprising a nucleotide sequence complementary to the nucleotide sequence of SEQ ID NO:32, and wherein said first polynucleotide encodes a protein which is able to catalyze the reaction of a substrate selected from the group consisting of a β-alanyl ester and a β-alanyl amide, with an amino acid or derivative thereof, to form a β-alanyl-amino acid or derivative thereof.

It is a further aspect of the present invention to provide the polynucleotide as described above, wherein said stringent conditions comprise washing at a salt concentration corresponding to 1×SSC and 0.1% SDS at 60° C.

It is a further aspect to provide a recombinant polynucleotide comprising the polynucleotide as described above.

It is a further aspect of the present invention to provide a cell transformed with the polynucleotide as described above.

### BRIEF DESCRIPTION OF DRAWINGS

FIG. 1 shows carnosine-forming activity and carnosine-degrading activity (U/mL) of RhDmpA3 (Rh3) and its homologs: BapA derived from Sphingosinicella microcystinivorans Y2 strain (Y2); DmpA derived from Pyrococcus horikoshii OT3 strain (PH); and DmpA derived from Aspergillus oryzae RIB40 strain (As), which were expressed in E. coli.

FIG. 2 shows rates (%) of carnosine-forming activity and carnosine-degrading activity of RhDmpA3 (Rh3) and its homologs: BapA derived from Sphingosinicella microcystinivorans Y2 strain (Y2); and DmpA derived from Aspergillus oryzae RIB40 strain (As), which were expressed in E. coli.

FIG. 3 shows carnosine-forming activity (%) of a purified RhDmpA enzyme when various β-alanine esters and a β-alanine amide were used as substrates. Abbreviations are as follows: β-AlaOMe: β-alanine methyl ester; β-AlaOEt: β-alanine ethyl ester; β-AlaOtBu: (3-alanine tert-butyl ester; β-AlaOBzl: β-alanine benzyl ester; and β-AlaNH₂: β-alanine amide.

FIG. 4 shows carnosine yield (vs Ala-%) of the purified RhDmpA enzyme when various β-alanine esters and β-alanine amide were used as the substrate. Abbreviations are the same as in FIG. 3.

### DETAILED DESCRIPTION OF THE EXEMPLARY EMBODIMENTS

### 1. Methods for Producing a β-Alanyl-Amino Acid or Derivative Thereof

In a method for producing a β-alanyl-amino acid or derivative thereof, the β-alanyl-amino acid (dipeptide) or derivative thereof is formed or produced from a β-alanyl ester or a β-alanyl amide, and an amino acid or derivative thereof, in the presence of an enzyme that has a certain activity. That is, a β-alanyl-amino acid or derivative thereof is formed or produced from a β-alanyl ester or a β-alanyl amide, and an amino acid or derivative thereof, in the presence of an enzyme or an enzyme-containing product that is able to catalyze formation or production of the β-alanyl-amino acid or derivative thereof from a β-alanyl ester or a β-alanyl amide, and an amino acid or derivative thereof.

The enzyme or the enzyme-containing product can mean an enzyme or an enzyme-containing product that substantially has an ability or an activity to catalyze a condensation reaction of a β-alanyl ester or a β-alanyl amide, and an amino acid or derivative thereof.

The ability to form or produce a β-alanyl-amino acid or derivative thereof from a β-alanyl ester or a β-alanyl amide, and an amino acid or derivative thereof, can include, for example, the ability to form or produce a β-alanyl-amino acid or derivative thereof from a β-alanyl ester and an amino acid or derivative thereof, the ability to form a β-alanyl-amino acid or derivative thereof from a β-alanyl amide and an amino acid or derivative thereof, the ability to form a β-alanyl-amino acid from a β-alanyl ester or a β-alanyl amide and an amino acid, the ability to form a derivative of a β-alanyl-amino acid from a β-alanyl ester or a β-alanyl amide and a derivative of an amino acid, and the ability to form a β-alanyl-amino acid from a β-alanyl ester and an amino acid.

The structures of the β-alanyl ester or the β-alanyl amide, and structures of the amino acid or derivative thereof, are not particularly limited as long as the reaction can be catalyzed by the enzyme.

Specifically, the β-alanyl ester that can be used as the substrate in the enzymatic reaction can be a compound represented by formula I: ₂HNCH₂CH₂CO—OR, and R can represent a substituted or unsubstituted hydrocarbon group.

Examples of the hydrocarbon group can include an alkyl group (e.g., a C₁₋₆ alkyl group such as methyl, ethyl, propyl, butyl, isobutyl, sec-butyl, tert-butyl, pentyl or hexyl) and a cycloalkyl group (e.g., a C₃₋₆ cycloalkyl group such as cyclopropyl, cyclobutyl, cyclopentyl or cyclohexyl).

Examples of the substituent on the substituted hydrocarbon group can include a halogen atom (e.g., fluorine, chlorine, bromine or iodine), a nitro group, a cyano group, a hydroxyl group, a carboxyl group, an amino group, the above alkyl groups, the above cycloalkyl groups, and an aryl group (e.g., phenyl, 1-naphthyl or 2-naphthyl).

The β-alanyl amide which can be used as the substrate in the enzymatic reaction can be a compound represented by formula II: ₂HNCH₂CH₂CO—NR₁R₂, and R₁ and R₂ are the same or different, and may represent a hydrogen atom or a substituted or unsubstituted hydrocarbon group. Here, the hydrocarbon group and the substituent can be the same as those described above.

The amino acid which can be used as the substrate in the enzymatic reaction can be a compound having both an amino group and a carboxyl group, and include, for example, histidine, alanine, valine, phenylalanine, lysine, arginine, aspartic acid, glutamic acid, glycine, asparagine, glutamine, threonine, leucine, isoleucine, proline, tyrosine, tryptophan, serine, cysteine and methionine. Examples of the amino acid can include an α-amino acid, a β-amino acid, and a γ-amino acid, and the α-amino acid and the β-amino acid are particular examples. The amino acid can be either an L-isomer or a D-isomer, but the L-amino acid is a particular example.

The amino acid derivative that can be used as the substrate in the enzymatic reaction can be a derivative that has been modified to retain the amino group. The amino acid derivative can be, for example, a derivative having a modification of a side chain of the above amino acid, or a derivative having a substitution of the carboxyl group of the above amino acid with a hydrogen atom or the above “substituent” (e.g., histamine having the structure in which the carboxyl group of histidine is substituted with a hydrogen atom). Examples of the derivative having a modification of a side chain of the above amino acid can include a compound in which the side chain of the above amino acid is substituted with the above “substituent” and a compound in which any atom (e.g., a hydrogen atom) or any group in the side chain of the above amino acid is substituted with the above “substituent.”

In one embodiment, the production method can be a method for producing β-alanyl-histidine. The method for producing β-alanyl-histidine or carnosine forms or produces β-alanyl-histidine from a β-alanyl ester and histidine in the presence of an enzyme that has a certain carnosine-forming activity. That is, the method for producing carnosine forms or produces β-alanyl-histidine from a β-alanyl ester and histidine with an enzyme or an enzyme-containing product that has the ability to form or produce β-alanyl-histidine from a β-alanyl ester and histidine. The enzyme or the enzyme-containing product that has the ability to form β-alanyl-histidine from a β-alanyl ester and histidine can refer to an enzyme or an enzyme-containing product that substantially has an ability or an activity to catalyze a condensation reaction of a β-alanyl ester and histidine, which is represented by the following chemical formula.

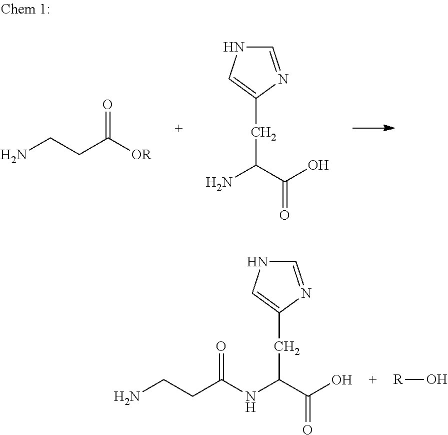

In this reaction, R can represent a substituted or unsubstituted hydrocarbon group. The hydrocarbon group and the substituent in the substituted hydrocarbon group can be the same as those described above.

The method of allowing the enzyme or the enzyme-containing product to act upon a β-alanyl ester or a β-alanyl amide and an amino acid or derivative thereof can include mixing the enzyme or the enzyme-containing product with the β-alanyl ester or the β-alanyl amide and the amino acid or derivative thereof. More specifically, the enzyme or the enzyme-containing product can be added to a solution containing the β-alanyl ester or the β-alanyl amide and the amino acid or derivative thereof, and the reaction is allowed to proceed. When a microorganism producing the enzyme is used as the enzyme-containing product, this microorganism can be cultured under conditions to produce and accumulate the enzyme in the microorganism or culture medium and the β-alanyl ester or the β-alanyl amide and the amino acid or derivative thereof can be added to the culture medium. This method can be used in addition to the method of adding the microorganism producing the enzyme to the above solution and allowing the reaction to proceed. The β-alanyl-amino acid or derivative thereof that is produced can be collected by standard methods and further purified, if necessary.

The enzyme-containing product can be a mixture containing the enzyme. The enzyme-containing product can further include one or more other substances. Specifically, the mixture containing the enzyme produced during, for example cultivation of the microorganism, or a mixture obtained by further subjecting the mixture to an aftertreatment as needed, can be used as the enzyme-containing product. Other substances that can be used as an enzyme-containing product can include cells and cellular debris of the microorganism in addition to the substances found in the culture medium. The aftertreatment can include recovery of the medium or recovery of microbial cells from the culture medium after completion of the cultivation; disruption, bacteriolysis and lyophilization of the microbial cells; partial removal of the other substances by crude purification and the like; immobilization of the enzyme or an entity (e.g., cell) containing the enzyme by a covalent bond method, an adsorption method or an inclusion method; and combinations thereof. Depending on the chosen microorganism, some microbial cells are lysed in the culture medium during the cultivation. Thus, in this case, the culture supernatant can also be utilized as the enzyme-containing product.

As the microorganism containing the enzyme, a wild-type strain, or a modified strain in which the present enzyme is expressed can be used. The microorganism is not limited to a microbial cell, but a treated microbial cell such as a microbial cell treated with acetone and a lyophilized microbial cell can also be used, or a immobilized microbial cell and a immobilized treated microbial cell obtained by the covalent bond method, the adsorption method or the inclusion method can be used.

When a wild-type strain which can produce the enzyme that has an activity to form a β-alanyl-amino acid or derivative thereof is used, such a strain is suitable because it can more simply produce the β-alanyl-amino acid or derivative thereof since it is not necessary to produce a genetically modified strain. On the other hand, if a mutant strain modified to produce large amounts of the enzyme that has the activity to form a β-alanyl-amino acid or derivative thereof (e.g., a genetically modified strain transformed to massively express a gene of the enzyme) is used, large amounts of the β-alanyl-amino acid or derivative thereof can be quickly and efficiently produced. That is, a microorganism transformed so that it is capable of expressing the enzyme that has the activity to form a β-alanyl-amino acid or derivative thereof (e.g., a protein described later), and a microorganism transformed so that it is capable of expressing a gene which encodes the enzyme (e.g., a polynucleotide described later) can also be used. The wild-type strain or the mutant strain can be cultured in the medium to produce and accumulate the enzyme in the medium and/or the microorganism, and the enzyme can be mixed with a β-alanyl ester or a β-alanyl amide and an amino acid or derivative thereof to form the β-alanyl-amino acid or derivative thereof.

When the culture, cultured microbial cell, washed microbial cell, or treated microbial product obtained by disrupting or lysing the microbial cell are used, an enzyme which degrades β-alanyl-amino acid or derivatives thereof without being involved in the formation of the β-alanyl-amino acid or derivative thereof is often present. Therefore, a metal protease inhibitor such as ethylenediamine tetracetic acid (EDTA) can be added. The amount to be added can be appropriately determined, and is typically in the range of, for example, 0.1 mM to 300 mM, and in another example, 1 mM to 100 mM.

The amount of the enzyme or the enzyme-containing product to be used can be an amount that exerts the objective effect (an effective amount). A person skilled in the art can easily determine this effective amount by a simple preliminary experiment. For example, the effective amount can be about 0.01 to 100 units (U) when the enzyme is used, and about 0.1 to 500 g/L when a washed microbial cell is used. One unit is the amount of the enzyme which forms 1 μmol of a β-alanyl-amino acid (e.g., β-alanyl-histidine) or derivative thereof per minute when the reaction is performed using 100 mM borate buffer containing 50 mM β-alanyl ester or β-alanyl amide and an amino acid (e.g., histidine) or derivative thereof at 25° C.

The amino acid or derivative thereof to be used for the reaction can be either an L-isomer or a D-isomer, but the L-amino acid or derivative thereof is a particular example.

The concentration of the starting material β-alanyl ester or the β-alanyl amide and the amino acid or derivative thereof can be 1 mM to 2 M and, in another example, 20 to 600 mM. If the reaction is inhibited when the concentration of the substrate is high, these substrates can be added sequentially so that the concentration does not inhibit the reaction.

The reaction temperature at which β-alanyl-amino acid or a derivative thereof is formed can be 5 to 60° C., and in another example, can be 10 to 40° C. The reaction pH at which β-alanyl-amino acid or derivative thereof can be formed can be pH 5 to 12, and in another example, pH 6 to 11. The produced β-alanyl-amino acid or derivative thereof can be crystallized by removing the enzymatic microbial cells and desalting with a resin and the like, followed by adding alcohol (methanol, ethanol, etc). For example, highly purified carnosine crystal can be precipitated by mixing an aqueous solution of crude L-carnosine containing impurities with alcohol such as methanol, adding L-carnosine seed crystal to the resulting mixed solution, and maturing at 30 to 80° C. for 1 to 10 hours, followed by further adding alcohol to the mixed solution (JP 2007-31328 A).

### 2. Microorganisms

Microorganisms that have an ability to form a β-alanyl-amino acid or derivative thereof from a β-alanyl ester or a β-alanyl amide and an amino acid or derivative thereof can be used without particular limitation. The microorganism that has an ability to form a β-alanyl-amino acid or a derivative thereof from a β-alanyl ester or a β-alanyl amide and an amino acid or derivative thereof can include microorganisms belonging to the genera Rhodotorula, Tremella, Candida, Cryptococcus, Erythrobasidium, Sphingosinicella, Pyrococcus, and Aspergillus. Specific examples thereof include Rhodotorula sp, Rhodotorula minuta, Tremella encephala, Candida mogii, Cryptococcus flavus, Rhodotorula marina, Rhodotorula aurantiaca, Erythrobasidium hasegawianum, Sphingosinicella microcystinivorans, Pyrococcus horikoshii, Aspergillus oryzae, and the like. Particular examples can include the following microbial strains, which were selected by determining microorganisms which can produce the enzyme which produces a β-alanyl-amino acid or derivative thereof with high yield from a β-alanyl ester or a β-alanyl amide and an amino acid or derivative thereof. Among the following microorganisms, (1), (2), (5), (11) and (15) have a particularly high activity.

(1) Rhodotorula minuta IFO0932 (Y129, AJ5019)

(2) Rhodotorula minuta IFO0879 (Y127, AJ5014)

(3) Tremella encephala IFO09293 (Y152, AJ14156, IFO0412)

(4) Rhodotorula minuta IFO0387 (Y-33-4, Y234, AJ4862)

(5) Candida mogii IFO0436 (I.G.C.3442, Y246, AJ5104)

(6) Cryptococcus flavus Y-33-8 IFO0710 (No. 41, AJ4864)

(7) Rhodotorula minuta K-38 (No. 50, AJ4873)

(8) Rhodotorula minuta KN-35 (No. 51, AJ4874, CBS5706, IFO1434)

(9) Rhodotorula minuta KN-36 CBS5695 (No. 52, AJ4875, IFO1435)

(10) Rhodotorula minuta AY-24 AJ4957 (No. 59)

(11) Rhodotorula sp AY-30 AJ4958 (No. 60) (FERM BP-11120)

(12) Rhodotorula marina NP-2-10 (No. 62, AJ4965)

(13) Rhodotorula aurantiaca IFO0754 (No. 65, AJ5011)

(14) Rhodotorula aurantiaca 68-254 AJ5119 (No. 74)

(15) Erythrobasidium hasegawianum IFO1058 (No. 92, AJ5228)

(16) Sphingosinicella microcystinivorans Y2 (JCM13185)

(17) Pyrococcus horikoshii OT3 (JCM9974, RDB5990) and

(18) Aspergillus oryzae RIB40 (NBRC G07-138-010)

Rhodotorula sp AY-30 AJ4958 (No. 60) was deposited on Nov. 6, 2007 under the accession number FERM P-21429 to the International Patent Organism Depositary (IPOD), National Institute of Advanced Industrial Science and Technology (AIST) (Chuo No. 6, Higashi 1-1-1, Tsukuba City, Ibaraki Pref., Japan), and was converted to an International Deposit on Apr. 24, 2009. The deposit number FERM BP-11120 was given to this strain, which can be obtained from IPOD.

Among the above strains, the strains having an IFO number were initially deposited to the Institute for Fermentation Osaka (17-85 Juso-honmachi 2-chome, Yodogawa-ku, Osaka, Japan), and subsequently transferred to the Biological Resource Center (NBRC) in Department of Biotechnology, National Institute of Technology and Evaluation (Kazusakamatari 2-5-8, Kisarazu-City, Chiba Pref., Japan) in 2003. Thus, they can be obtained from NBRC.

Among the above strains, the strains having the CBS number can be obtained from Centraalbureau voor Schimmelcultures (Uppsalalaan 8 3584 CT Utrecht The Netherlands).

Among the above strains, the strains having the JCM number can be obtained from Incorporated Administrative Agency RIKEN, Bioresource Center (Hirosawa 2-1, Wako-City, Saitama Pref., Japan).

For these microorganisms, a wild-type strain or a mutant strain can be used, and a modified strain derived by a genetic technique such as cell fusion or gene engineering can also be used.

To obtain a microbial cell of such a microorganism, the microorganism may be cultured and grown in an appropriate medium. The medium is not particularly limited as long as the microorganism can grow, and can be an ordinary medium containing a carbon source, a nitrogen source, a phosphorous source, a sulfur source, inorganic ions, and if necessary, organic nutrient sources.

For example, for the carbon source, anything which can be used by the above microorganism can be used. Specifically, sugars such as glucose, fructose, maltose and amylose, alcohols such as sorbitol, ethanol and glycerol, organic acids such as fumaric acid, citric acid, acetic acid and propionic acid and salts thereof, hydrocarbons such as paraffin, and mixtures thereof can be used.

For the nitrogen source, ammonium salts of inorganic acids such as ammonium sulfate and ammonium chloride, ammonium salts of organic acids such as ammonium fumarate and ammonium citrate, phosphate salts such as monopotassium phosphate and dipotassium phosphate, sulfate salts such as magnesium sulfate, nitrate salts such as sodium nitrate and potassium nitrate, organic nitrogen compounds such as peptone, yeast extracts, meat extracts and corn steep liquor, and mixtures thereof can be used.

In addition, nutrient sources such as inorganic salts, trace metal salts and vitamins, which are typically used for the medium can be appropriately mixed and used.

The cultivation conditions are not particularly limited, and can be performed under aerobic conditions for about 12 to 48 hours while appropriately controlling pH and the temperature in the range of pH 5 to 8 and the temperature at 15 to 40° C.

### 3. Enzyme

The enzyme that has an ability to form a β-alanyl-amino acid or derivative thereof from a β-alanyl ester or a β-alanyl amide and an amino acid or derivative thereof can be used. As long as the enzyme has such an activity, its origin and the method for acquiring it are not limited. Hereinafter, an exemplary enzymatic protein, its purification, utilization of gene engineering techniques, and the like will be described.

(3-1) Protein

The protein can be obtained from a microorganism that has an ability to form a β-alanyl-amino acid or derivative thereof from a β-alanyl ester or a β-alanyl amide, and an amino acid or derivative thereof. For example, exemplary microorganisms include those described above, i.e., microorganisms belonging to a genus such as Rhodotorula, Tremella, Candida, Cryptococcus, Erythrobasidium, Sphingosinicella, Pyrococcus and Aspergillus can be utilized. More specifically, the microbial strains (1) to (18) described above can be included.

Exemplary methods of isolating and purifying the protein from the microorganism described above will be described.

First, the microbial cells can be disrupted by a physical method such as ultrasonic disruption or an enzymatic method using a cell wall dissolving enzyme, and the insoluble fraction can be removed by centrifugation and the like, to prepare a microbial cell extract solution. The protein can be purified by fractionating the microbial cell extract solution thus obtained by a combination of typical protein purification methods, such as ammonium sulfate fractionation, dialysis, anion exchange chromatography, hydrophobic chromatography, cation exchange chromatography, gel filtration chromatography, and the like.

Exemplary carriers for anion exchange chromatography can include Q-Sepharose FF 26/10, 16/10, HP, and Mono-Q HR5/5 (all supplied from Pharmacia (GE health Care Bioscience)). When the extract solution containing the enzyme is passed through a column packed with this carrier, the enzyme can be collected from a non-adsorbed fraction at pH 7.6.

Exemplary carriers for the hydrophobic chromatography can include PhenylSepharose HP 16/10 (supplied from Pharmacia (GE health Care Bioscience)). The extract solution containing the enzyme is passed through a column packed with this carrier to adsorb the enzyme to the column, which is then washed, and the enzyme is then eluted using a buffer with a high salt concentration. At that time, the salt concentration can be increased stepwise or a concentration gradient can be applied.

Exemplary carriers for the cation chromatography can include Monos HR (supplied from Pharmacia (GE health Care Bioscience)). The extract solution containing the enzyme is passed through a column packed with this carrier to adsorb the present enzyme to the column, which is then washed, and the enzyme is then eluted using a buffer with a high salt concentration. At that time, the salt concentration can be increased stepwise or a concentration gradient can be applied.

Exemplary carriers for the gel filtration chromatography can include Superdex 200 pg and Sephadex 200 pg 16/10 (both supplied from Pharmacia (GE health Care Bioscience)).

The fraction containing the objective enzyme can be identified by measuring the activity of forming a β-alanyl-amino acid or derivative thereof in each fraction by methods shown in the Examples described later during the above purification manipulation. An internal amino acid sequence of the objective enzyme purified as above is shown in SEQ ID NOS:9 and 10.

The protein can have the amino acid sequence of SEQ ID NO:8 as an N-terminal amino acid sequence, and can include the amino acid sequence of SEQ ID NO:9 as an internal amino acid sequence, as well as homologs of these sequences. More specifically proteins such as those listed as the following (A) to (L) can be included.

(A) a protein having the amino acid sequence of SEQ ID NO:3;

(B) a protein having the amino acid sequence of SEQ ID NO: 3, but which includes one or more substitutions, deletions and/or insertions of one or several amino acids and which maintains the activity to form the β-alanyl-amino acid or derivative thereof from the β-alanyl ester or the β-alanyl amide and the amino acid or derivative thereof;

(C) a protein having the amino acid sequence of SEQ ID NO:5;

(D) a protein having the amino acid sequence of SEQ ID NO: 5, but which includes one or more substitutions, deletions and/or insertions of one or several amino acids and which maintains the activity to form the β-alanyl-amino acid or derivative thereof from the β-alanyl ester or the β-alanyl amide and the amino acid or derivative thereof;

(E) a protein having the amino acid sequence of SEQ ID NO:7;

(F) a protein having the amino acid sequence of SEQ ID NO: 7, but which includes one or more substitutions, deletions and/or insertions of one or several amino acids and maintains the activity to form the β-alanyl-amino acid or derivative thereof from the β-alanyl ester or the β-alanyl amide and the amino acid or derivative thereof;

(G) a protein having the amino acid sequence of SEQ ID NO:21;

(H) a protein having the amino acid sequence of SEQ ID NO:21, but which includes one or more substitutions, deletions and/or insertions of one or several amino acids and maintains the activity to form the β-alanyl-amino acid or derivative thereof from the β-alanyl ester or the β-alanyl amide and the amino acid or derivative thereof;

(I) a protein having the amino acid sequence of SEQ ID NO:23;

(J) a protein having the amino acid sequence of SEQ ID NO: 23, but which includes one or more substitutions, deletions and/or insertions of one or several amino acids and maintains the activity to form a β-alanyl-amino acid or derivative thereof from a β-alanyl ester or a β-alanyl amide and an amino acid or derivative thereof;

(K) a protein having the amino acid sequence of SEQ ID NO:25; and

(L) a protein having the amino acid sequence of SEQ ID NO: 25, but which includes one or more substitutions, deletions and/or insertions of one or several amino acids and maintains the activity to form the β-alanyl-amino acid or derivative thereof from the β-alanyl ester or the β-alanyl amide and the amino acid or derivative thereof.

The proteins having the amino acid sequences of SEQ ID NOs: 3, 5 and 7 were newly isolated from Rhodotorula minuta IFO0932 (Y129, AJ5019), and their amino acid sequences were identified. The proteins having the amino acid sequences of SEQ ID NOs: 3, 5 and 7 are related in that they all include at least portions of the amino acid sequence of SEQ ID NO: 7.

The protein can include substantially the same amino acid sequence as those described in any of SEQ ID NOs: 3, SEQ ID NO: 5, SEQ ID NO: 7, SEQ ID NO: 21, SEQ ID NO: 23 and SEQ ID NO: 25. Specifically, the proteins in above (B), (D), (F), (H), (J) and (L) may be included.

The number meant by the term “several” can vary depending on the position of the objective amino acid in the three-dimensional structure of the protein, and the kind of amino acid residue, and can be in a range such that the three-dimensional structure and the activity of the protein are not significantly impaired, and in one example, can be 1 to 50, in another example, can be 1 to 30, and in yet another example, can be 1 to 10. The sequence having one or several amino acid mutations can be 70% or more, 80% or more, 90% or more, 95% or more, or even 97% or more homologous or identical to a sequence which has no mutations. However, the protein which includes one or more substitutions, deletions and/or insertions of one or several amino acids such as those in (B), (D), (F), (H), (J) and (L) can retain the enzymatic activity at about half or more, 80% or more, or even 90% or more as compared to the protein which has no mutations when at 50° C. at pH 8. For example, using protein (B) as an example, this protein can retain the enzymatic activity at about a half or more, 80% or more, or even 90% or more as compared to a protein having the amino acid numbers 14 to 340 in the sequence of SEQ ID NO:3 at 50° C. at pH 8.

The homology or the identity can be obtained by calculating the number of amino acid residues in the full length sequence and designating this number as the denominator, and designating the number of identical amino acid residues when comparing two sequences the numerator, and multiplying the calculated value by 100. The homology or the identity can also be calculated using “Genetyx” (GENETIX Ltd.) using default parameters.

The mutation of the of the sequence as exemplified in protein (B) as explained above, can be obtained, for example, by designing the amino acid sequence of protein (A), and then modifying it by site-specific mutagenesis so that the amino acid at the particular pre-determined positions are substituted, deleted and/or inserted, and then expressing the nucleotide sequence corresponding to this amino acid sequence.

The substitution, deletion, insertion and/or the like of nucleotides as described above can include naturally occurring mutations such as differences that occur due to the type or strain of the microorganism. Proteins similar to the proteins described in any of SEQ ID NOS: 3, 5, 7, 21, 23 and 25 can be obtained by expressing a polynucleotide encoding the amino acid sequence having the mutation(s) described above in an appropriate cell and examining the activity of the expressed enzyme product.

Substitutions can be conservative substitutions. “Conservative substitutions” can mean that a certain amino acid is substituted with an amino acid that has a analogous side chain. Amino acids having analogous side chains, or families of amino acids, are well-known in the art. Examples of such families include amino acids having a basic side chain (e.g., lysine, arginine or histidine), amino acids having an acidic side chain (e.g., aspartic acid or glutamic acid), amino acids having a non-charged polar side chain (e.g., glycine, asparagine, glutamine, serine, threonine, tyrosine or cysteine), amino acids having a nonpolar side chain (e.g., alanine, valine, leucine, isoleucine, proline, phenylalanine, methionine or tryptophan), amino acids having a β-position branched side chain (e.g., threonine, valine or isoleucine), amino acids having an aromatic side chain (e.g., tyrosine, phenylalanine, tryptophan or histidine), amino acids having a hydroxyl-containing side chain (e.g., alcoholic or phenolic) (e.g., serine, threonine or tyrosine), and amino acids having a sulfur-containing side chain (e.g., cysteine or methionine). Conservative substitutions include substitutions between aspartic acid and glutamic acid, substitutions among arginine, lysine and histidine, substitutions between tryptophan and phenylalanine, substitutions between phenylalanine and valine, substitutions among leucine, isoleucine and alanine, and substitutions between glycine and alanine.

(3-2) Preparation and Purification of the Polynucleotide, Recombinant Polynucleotide, and Transformant, and Purification of the Enzyme

(3-2-1) Polynucleotide Encoding the Protein

A polynucleotide encoding the protein described above is disclosed. The polynucleotide can encode the amino acid sequences as described above, and can include multiple nucleotide sequences encoding for a single amino acid sequence due to degeneracy of the nucleotide code. Specifically, it can include a polynucleotide having a nucleotide sequence encoding the protein such as those described above in (A) to (L).

Specific examples of the polynucleotide can include polynucleotides such as the following (a) to (n).

(a) a polynucleotide having the nucleotide sequence of nucleotide numbers 40 to 1239 of SEQ ID NO:1;

(b) a polynucleotide which hybridizes under stringent conditions with a polynucleotide having a nucleotide sequence which is complementary to the nucleotide sequence of nucleotide numbers 40 to 1239 of SEQ ID NO:1, and encodes a protein having an activity to form a β-alanyl-amino acid or derivative thereof from a β-alanyl ester or a β-alanyl amide and an amino acid or derivative thereof;

(c) a polynucleotide having the nucleotide sequence of nucleotide numbers 55 to 1239 of SEQ ID NO:1;

(d) a polynucleotide which hybridizes under stringent conditions with a polynucleotide having a nucleotide sequence which is complementary to the nucleotide sequence of nucleotide numbers 55 to 1239 of SEQ ID NO:1, and encodes a protein having an activity to form a β-alanyl-amino acid or derivative thereof from a β-alanyl ester or a β-alanyl amide and an amino acid or derivative thereof;

(e) a polynucleotide having the nucleotide sequence of nucleotide numbers 91 to 1239 of SEQ ID NO:1;

(f) a polynucleotide which hybridizes under stringent conditions with a polynucleotide having a nucleotide sequence which is complementary to the nucleotide sequence of nucleotide numbers 91 to 1239 of SEQ ID NO:1, and encodes a protein having an activity to form a β-alanyl-amino acid or derivative thereof from a β-alanyl ester or a β-alanyl amide and an amino acid or derivative thereof;

(g) a polynucleotide having the nucleotide sequence of SEQ ID NO:20;

(h) a polynucleotide which hybridizes under stringent conditions with a polynucleotide having a nucleotide sequence which is complementary to the nucleotide sequence of SEQ ID NO:20, and encodes a protein having an activity to form a β-alanyl-amino acid or derivative thereof from a β-alanyl ester or a β-alanyl amide and an amino acid or derivative thereof;

(i) a polynucleotide having the nucleotide sequence of SEQ ID NO:22;

(j) a polynucleotide which hybridizes under stringent conditions with a polynucleotide having a nucleotide sequence which is complementary to the nucleotide sequence of SEQ ID NO:22, and encodes a protein having an activity to form a β-alanyl-amino acid or derivative thereof from a β-alanyl ester or β-alanyl amide and an amino acid or the derivative thereof; (k) a polynucleotide having the nucleotide sequence of SEQ ID NO:24;

(l) a polynucleotide which hybridizes under stringent conditions with a polynucleotide having a nucleotide sequence complementary to the nucleotide sequence of SEQ ID NO:24, and encodes a protein having an activity to form β-alanyl-amino acid or derivative thereof from a β-alanyl ester or a β-alanyl amide and an amino acid or derivative thereof;

(m) a polynucleotide having the nucleotide sequence of SEQ ID NO:32; and

(n) a polynucleotide which hybridizes under stringent conditions with a polynucleotide having a nucleotide sequence complementary to the nucleotide sequence of SEQ ID NO:32, and encodes a protein having an activity to form a β-alanyl-amino acid or derivative thereof from a β-alanyl ester or a β-alanyl amide and an amino acid or derivative thereof.

The polynucleotides having the nucleotide sequences of SEQ ID NO:1 and SEQ ID NO:32 were isolated from Rhodotorula minuta IFO0932 (Y129, AJ5019). The nucleotide sequence of SEQ ID NO:1 contains three open reading frames (ORF), which are represented by SEQ ID NO:2, SEQ ID NO:4, and SEQ ID NO:6; that is, the nucleotide sequence of SEQ ID NO:1 is similar to the nucleotide sequence represented by SEQ ID NO:2, SEQ ID NO:4 and SEQ ID NO:6, which represent three different ORFs, respectively. These three ORFs represent nucleotide numbers 40 to 1239 of SEQ ID NO:1, which is equivalent to SEQ ID NO:2, nucleotide numbers 55 to 1239 of SEQ ID NO:1, which is equivalent to SEQ ID NO:4, and nucleotide numbers 91 to 1239 of SEQ ID NO:1, which is equivalent to SEQ ID NO:6. A protein encoded by this ORF will exhibit a high activity to form carnosine. The nucleotide sequence in SEQ ID NO:1 is common to each of the nucleotide sequences of SEQ ID NOS:2, 4 and 6. On the other hand, a polynucleotide having the nucleotide sequence of SEQ ID NO:32 is a genomic DNA containing the polynucleotide of SEQ ID NO:1 as an exon region, and also containing an intron region. Such a genomic DNA can express the proteins encoded by the above three ORFs by transforming a certain host cell (e.g., yeast) having a splicing ability, and can exhibit an activity to form a β-alanyl-amino acid or derivative thereof.

Various gene recombination techniques included below can be carried out in accordance with descriptions in Molecular Cloning, 2nd edition, Cold Spring Harbor press (1989), and the like.

The polynucleotide can be obtained from cDNA or genomic DNA, or a cDNA library or a genomic library of Rhodotorula minuta, and the like by PCR (polymerase chain reaction, see White, T. J. et al., Trends Genet., 5, 185 (1989)) or hybridization. Primers used for PCR can be designed based on the internal amino acid sequence of the enzyme purified as described in above (3). Since the nucleotide sequence of the gene encoding the enzyme (SEQ ID NO:1) is first described in accordance with the presently disclosed subject matter, the primers and probes for the hybridization can be designed based on this nucleotide sequence, and the polynucleotide can also be isolated using the probe. When primers having the sequences corresponding to a 5′-untranslated region and a 3-untranslated region are used as the primers for PCR, a full length coding region of the enzyme can be amplified. Specifically, a 5′-side primer can include a nucleotide sequence in the region upstream from the nucleotide number 40 of SEQ ID NO:1, a primer having a nucleotide sequence in the region upstream from the nucleotide number 55 of SEQ ID NO:1, and a primer having the nucleotide sequence in the region upstream from the nucleotide number 91. A 3′-side primer can include a primer having a sequence that is complementary to a nucleotide sequence in the region downstream from the nucleotide number 1239.

The primer can be synthesized using a DNA synthesizer Model 380B supplied from Applied Biosystems, and using phosphoamidite in accordance with standard methods (see Tetrahedron Letters (1981), 22, 1859). PCR can be performed using Gene Amp PCR System 9600 (supplied from PERKIN ELMER) and TaKaRa LA PCR in vitro Cloning Kit (supplied from Takara Shuzo Co., Ltd.) in accordance with methods suggested by each manufacturer.

The polynucleotide encoding the enzyme that can be used can include polynucleotides which are substantially the same as any of the ORFs of the polynucleotide of SEQ ID NO:1 or the polynucleotide of SEQ ID NO:32, or the polynucleotides of any of SEQ ID NO:20, SEQ ID NO:22 and SEQ ID NO:24. That is, a polynucleotide substantially similar to the polynucleotide as described above can be obtained by isolating a polynucleotide that hybridizes under stringent conditions with the polynucleotide having a nucleotide sequence complementary to any of the ORFs of SEQ ID NO:1, or the polynucleotide of SEQ ID NO:32, or the polynucleotide of any of SEQ ID NO:20, SEQ ID NO:22 or SEQ ID NO:24, or with the probe prepared from the same nucleotide sequence, and encodes a protein having a carnosine-forming activity.

The probe can be made based on the nucleotide sequence described in any of SEQ ID NO:1, SEQ ID NO:21, SEQ ID NO:23, SEQ ID NO:25 and SEQ ID NO:32 by standard methods. Using the probe, a polynucleotide that hybridizes to any of these sequences can be used to isolate the objective polynucleotide in accordance with the standard methods. For example, the DNA probe can be prepared by amplifying the nucleotide sequence cloned in a plasmid or a phage vector, cutting out an objective nucleotide sequence with restriction enzymes, and extracting it. Sites to be cut out can be controlled depending on the objective polynucleotide.

The “stringent conditions” can mean conditions where a so-called specific hybrid is formed, and non-specific hybrids are not formed. Examples can include conditions wherein a pair of polynucleotides with high homology, e.g., the polynucleotides which are 50% or more homologous, 80% or more homologous, 90% or more homologous, 95% or more homologous, and even 97% or more homologous hybridize to each other, whereas polynucleotides with homology lower than the above do not hybridize, or washing conditions used in an ordinary Southern hybridization, i.e., hybridization at salt concentrations equivalent to 1×SSC (sodium chloride/sodium citrate) and 0.1% SDS at 60° C., or 0.1×SSC and 0.1% SDS at 60° C. Another example of stringent conditions is a hybridization in 6×SSC at about 45° C. followed by one or two washings in 0.2×SSC and 0.1% SDS at 50 to 65° C. These genes that hybridize under such conditions include those in which a stop codon occurs in an internal region of a sequence and those that lose activity due to mutation of an active center. However, these can be easily removed by ligating them to a commercially available expression vector, expressing them in an appropriate host, and measuring the enzymatic activity of the expressed product by methods described herein.

In this regard, however, a polynucleotide having a nucleotide sequence that hybridizes under the stringent conditions as described above can retain the enzymatic activity at about a half or more, 80% or more, and even 90% or more as compared to a protein having the amino acid sequence encoded by the original nucleotide sequence, at 50° C. at pH 8. For example, and using sequence (b) as an example, the nucleotide sequence that hybridizes under stringent conditions with a polynucleotide having a nucleotide sequence which is complementary to the nucleotide sequence of the nucleotide numbers 40 to 1239 of SEQ ID NO: lcan encode a protein that can retain an enzymatic activity at about a half or more, 80% or more, and even 90% or more as compared to a protein having the amino acid sequence of SEQ ID NO:3 at 50° C. at pH 8.

The polynucleotide thus modified can be obtained by conventionally known mutagenesis. Such mutagenesis can include treating a polynucleotide encoding the enzyme, e.g., any polynucleotide of (a), (c), (e), (g), (i), (k) and (m), with hydroxylamine or the like in vitro, and treating Escherichia cells carrying a polynucleotide encoding the enzyme with a mutagen typically used for artificial mutation, such as ultraviolet ray, N-methyl-N′-nitro-N-nitrosoguanidine (NTG) or nitrous acid.

Three ORFs are included in SEQ ID NO:1 as described above (see SEQ ID NO: 2, 4 and 6), and the amino acid sequence encoded by each ORF is represented by SEQ ID NO:3, SEQ ID NO:5 or SEQ ID NO:7.

(3-2-2) Preparation of a Recombinant Polynucleotide (Expression Vector) and Transformants, as Well as Formation of the Enzyme

The enzyme (also referred to as an “enzyme which forms a β-alanyl-amino acid or derivative thereof” or a “carnosine-forming enzyme”) can also be formed by introducing the polynucleotide described above (see section 3-2-1) into an appropriate host to make a recombinant polynucleotide, and expressing the protein specified by the polynucleotide in the transformed cells (transformant).

As the host for expressing the protein specified by the polynucleotide, various prokaryotic cells including bacteria belonging to genus Escherichia such as Escherichia coli and cells from Bacillus subtilis, and various eukaryotic cells including Saccharomyces cerevisiae, Pichia stipitis and Aspergillus oryzae can be used.

The recombinant polynucleotide that can be used to transform the host can be prepared by inserting the objective polynucleotide into a vector chosen according to the type of host so that the polynucleotide is expressed. When the native promoter for the gene encoding the enzyme which forms a β-alanyl-amino acid or derivative thereof works in the host cell, the promoter can be used to express the polynucleotide. Other promoters can be used to express the polynucleotide if they work in the chosen host cell, and if necessary, can be ligated to the polynucleotide so that it is under the control of the promoter.

Exemplary transformation methods for introducing the recombinant polynucleotide into the host cell can include D. M. Morrison's method (Methods in Enzymology 68, 326 (1979)), or a method of increasing permeability of a polynucleotide by treating the recipient bacterial cell with calcium chloride (Mandel, M. and Higa, A., J. Mol. Biol., 53, 159 (1970)).

When producing a protein on a large scale using recombinant polynucleotide technology, inclusion bodies of the protein can be formed. These are caused by the association of the protein in the transformed cell. The advantages of this expression production method can include protection of the objective protein from protease digestion, and ready purification of the objective protein by disruption of the microbial cells, and subsequent centrifugation.

The protein inclusion bodies obtained in this way can be solubilized by a protein denaturing agent, and activity is regenerating mainly by eliminating the denaturing agent, so that correctly refolded and physiologically active proteins are recovered. There are many examples of such procedures, such as regenerating activity of human interleukin 2 (JP 61-257931 A).

To obtain an active protein from the protein inclusion body, a series of the manipulations such as solubilization and regenerating activity is required, and thus the manipulations are more complicated than those when directly producing an active protein. However, when a protein that affects the microbial cell growth is produced on a large scale in the microbial cells, the effects thereof may be inhibited by allowing the protein to form as an inclusion body of the inactive protein in the microbial cells.

The methods for producing the objective protein on a large scale using inclusion bodies include methods of expressing a protein alone under the control of a strong promoter, as well as methods of expressing the objective protein as a fusion protein with a protein known to be expressed in a large amount.

Hereinafter, the method of transforming Escherichia coli (E. coli) and producing the enzyme which forms a β-alanyl-amino acid or derivative thereof using this will be described and exemplified more specifically.

As the promoter for expressing the polynucleotide encoding the enzyme which forms a β-alanyl-amino acid or derivative thereof, a promoter which is typically used for producing a foreign protein in E. coli can be used, and examples thereof may include strong promoters such as the T7 promoter, lac promoter, trp promoter, trc promoter, tac promoter, and PR promoter and PL promoter of lambda phage. As a vector, pUC19, pUC18, pBR322, pHSG299, pHSG298, pHSG399, pHSG398, RSF1010, pMW119, pMW118, pMW219, pMW218 and the like can be used. In addition, a phage polynucleotide can also be utilized as the vector. Furthermore, an expression vector that contains the promoter and can express the inserted polynucleotide sequence can also be used.

In order to produce the enzyme which forms a β-alanyl-amino acid or derivative thereof as an inclusion body of the fusion protein, a gene encoding another protein, such as a hydrophilic peptide, is ligated to an upstream or downstream portion of the gene encoding the enzyme which forms a β-alanyl-amino acid or derivative thereof in order to produce a fusion protein gene. A gene encoding another protein can be one which increases the accumulation amount of the fusion protein and enhances solubility of the fusion protein after denaturation and regeneration, and examples thereof include the T7 gene 10, β-galactosidase gene, dehydrofolate reductase gene, interferon γ gene, interleukin-2 gene, prochymosin gene and the like.

These genes can be ligated to the gene encoding the enzyme which forms a β-alanyl-amino acid or derivative thereof so that reading frames of codons are matched. Such a ligation may be performed at an appropriate restriction enzyme site, or by utilization of a synthetic polynucleotide with an appropriate sequence.

In order to augment the production amount, a terminator, i.e., a transcription termination sequence, can be ligated downstream of the gene encoding the fusion protein. This terminator can include the rrnB terminator, T7 terminator, fd phage terminator, T4 terminator, terminator of tetracycline resistant gene and the terminator of E. coli trpA gene.

As the vector to introduce the gene encoding the enzyme or the fusion protein which forms a β-alanyl-amino acid or derivative thereof and another protein into E. coli, so-called multiple copying types can be used, including plasmids having a replication origin derived from ColE1, such as pUC type plasmids, pBR322 type plasmids or derivatives thereof. Here, the “derivative” can mean one in which plasmids are modified by substitution, deletion and/or insertion of nucleotides. The modification can also include modification by mutagenic treatments by mutagenic agents and UV irradiation or natural mutation.

The vector can have a marker such as an ampicillin resistant gene for selection of the transformant. As such a plasmid, expression vectors carrying strong promoters are commercially available (pUC types (supplied from Takara Shuzo Co., Ltd.), pPROK types (supplied from Clontech), pKK233-2 (supplied from Clontech) and the like).

The recombinant polynucleotide can be obtained by ligating the promoter, the gene encoding the enzyme or the fusion protein which forms a β-alanyl-amino acid or derivative thereof and another protein, and in some cases the terminator, in this order to obtain a polynucleotide fragment, and further ligating the resulting polynucleotide fragment to the vector polynucleotide.

Using the resulting recombinant polynucleotide, E. coli is transformed. Cultivation of this E. coli results in expression and production of the enzyme or the fusion protein which forms a β-alanyl-amino acid or derivative thereof and another protein. The chosen host can be strains that are usually employed for the expression of foreign genes, and E. coli JM109 strain is one example. Methods for performing transformation and methods of selecting the transformant are described in Molecular Cloning, 2nd edition, Cold Spring Harbor Press (1989) and the like.

In the case of expressing the enzyme as a fusion protein, the fusion protein can be composed so as to be able to cleave the enzyme which forms a β-alanyl-amino acid or derivative thereof therefrom using a restriction protease which recognizes a sequence of blood coagulation factor Xa, kallikrein, or the like, which is not present in the enzyme.

The production media can include media typically used for culturing E. coli, such as M9-casamino acid medium and LB medium. Culture conditions and production induction conditions can be appropriately selected depending on types of the vector marker, the promoter, the host bacterium and the like.

The enzyme or the fusion protein which forms a β-alanyl-amino acid or derivative thereof and another protein may be recovered by the following method: when the enzyme or the fusion protein thereof is solubilized in the microbial cells, the microbial cells may be collected and then disrupted or lysed, to obtain a crude enzyme solution. If necessary, the enzyme or the fusion protein thereof can be further purified in accordance with ordinary methods such as precipitation, filtration and column chromatography. In this case, the purification can also be performed utilizing an antibody against the enzyme that forms a β-alanyl-amino acid or derivative thereof or the fusion protein thereof.

When the inclusion body of the protein is formed, this can be solubilized with the denaturing agent. The inclusion body can be solubilized together with the microbial cells. However, considering the following purification process, the inclusion body can be removed before solubilization. Collection of the inclusion body from the microbial cells can be performed in accordance with conventionally and publicly known methods. For example, the microbial cells are broken, and the inclusion body is collected by centrifugation and the like. The denaturing agent which solubilizes the protein inclusion body may include guanidine-hydrochloric acid (e.g., 6 M, pH 5 to 8) and urea (e.g., 8 M

As a result of removal of the denaturing agent by dialysis and the like, the active protein can be regenerated. Dialysis solutions can include tris hydrochloric acid buffer and phosphate buffer. The concentration thereof can be 20 mM to 0.5 M, and the pH can be 5 to 8.

The protein concentration at the regeneration step can be kept at about 500 μg/ml or less. In order to inhibit self-crosslinking of the regenerated enzyme which forms a β-alanyl-amino acid or derivative thereof, the dialysis temperature can be kept at 5° C. or below. Methods for removing the denaturing agent other than by dialysis can include by dilution or ultrafiltration. The regeneration of the activity can be expected by using any of these methods.

As described above, the method for producing the enzyme which forms a β-alanyl-amino acid or derivative thereof has been described using transformed E. coli as an example. Yeast can also be transformed with the gene encoding the enzyme that forms a β-alanyl-amino acid or derivative thereof. The yeast is easily cultured, and it is safe because the yeast has been traditionally used for producing food products. When transforming yeast, a known promoter can be utilized. Examples of such promoters can include CYC1, HIS3, GAL1, GAL10, ADH1, PGK, PHO5, TRP1, URA3, LEU2, Mal, ENO, TPI and AOX1. An appropriate terminator such as TPI terminator can also be utilized.

Specifically, any multicopy-type vector (YEp type), single copy-type vector (YCp type), or a chromosomal integration-type vector (YIp type) can be utilized used when introducing into the yeast. The expression vectors such as YEp24, YCp50 and YIp5 are known as YEp type vector, YCp type vector and YIp type vector, respectively, and can also be utilized to transform yeast.

For example, the recombinant polynucleotide can be obtained by ligating the promoter, the gene encoding the enzyme which forms a β-alanyl-amino acid or derivative thereof or the fusion protein of the enzyme which forms a β-alanyl-amino acid or derivative thereof and another protein, and in some cases the terminator, in this order to obtain a polynucleotide fragment, and further ligating the resulting polynucleotide fragment to the vector polynucleotide.

Using the resulting recombinant polynucleotide, the yeast can be transformed. Cultivation of this yeast results in expression and production of the enzyme which forms a β-alanyl-amino acid or derivative thereof or the fusion protein of the enzyme which forms a β-alanyl-amino acid or derivative thereof and another protein. The chosen host can be strains that are usually employed for an expression of foreign genes, and for example, a yeast belonging to genus Saccharomyces or Pichia can be used. The yeast can be cultured in a known medium such as SD medium, YPD medium, YPAD medium or SC medium. Methods for transformation, methods of selecting the transformant and methods of culturing the resulting transformant are described in Molecular Cloning, 2nd edition, Cold Spring Harbor Press (1989) and the like. The enzyme which forms a β-alanyl-amino acid or derivative thereof or the fusion protein of the enzyme which forms a β-alanyl-amino acid or derivative thereof and another protein can be recovered in the same manner as in the methods aforementioned for E. coli.

### EXAMPLES

The present invention will be illustrated in more detail with reference to the following Examples, but the invention is not limited thereto. Carnosine was quantified using high performance liquid chromatography in the Examples.

1. Column: Inertsil ODS-3 (4.6×250 mm), column temperature: 40° C., eluant: 0.1 M KH₂PO₄—H₃PO₄ (pH 2.1)/CH₃CN=10/2, flow rate: 0.7 mL/min, and detection: UV 210 nm.

β-Ala-X, other than carnosine, was quantified using the high performance liquid chromatography.

2. Column: Inertsil ODS-3 (4.6×250 mm), column temperature: 40° C., eluant: 0.1M NaH₂PO₄—H₃PO₄ (pH 2.1)/CH₃OH=2/1, flow rate: 1.0 mL/min, and detection: UV 210 nm.

### Example 1

### Formation of Carnosine from β-AlaOMe and L-His by Microbial Cell Reaction in an Active Microorganism

Rhodotorula minuta IFO0879, Rhodotorula minuta IFO0932, Tremella encephala IFO9293, Rhodotorula minuta IFO0387, Candida mogii IFO0436, Cryptococcus flavus Y-33-8 IFO0710, Rhodotorula minuta K-38 AJ4873, Rhodotorula minuta KN-35 CBS5706, Rhodotorula minuta KN-36 CBS5695, Rhodotorula minuta AY-24 AJ4957, Rhodotorula sp. AY-30 AJ4958 (FERM P-21429), Rhodotorula marina NP-2-10 AJ4965, Rhodotorula aurantiaca IFO0754, Rhodotorula aurantiaca 68-254 AJ5119 and Erythrobasidium hasegawianum IFO1058 were applied to plate media containing 10 g/L of glucose, 3 g/L of yeast extract, 3 g/L of malt extract, 5 g/L of peptone and 15 g/L of agar, and cultured at 25° C. for 2 days.

One loopful of the resulting microbial cells was inoculated into 50 mL of liquid medium containing 10 g/L of glucose, 3 g/L of yeast extract, 3 g/L of malt extract and 5 g/L peptone in a 500 mL Sakaguchi flask, which was then cultured with shaking at 25° C. for 24 hours. After the cultivation, the microbial cells were collected from the culture by centrifugation, washed with 25 mL of saline and suspended in saline to prepare a microbial cell suspension.

This microbial cell suspension (100 μL) and 100 μL of a substrate solution containing 200 mM β-AlaOMe, 200 mM L-His, 20 mM EDTA and 200 mM borate buffer (pH 9.0) were mixed and allowed to react at 25° C. for 15 hours. After completion of the reaction, the amount of carnosine was measured (Table 1).

TABLE 1
Table 1. An amount of carnosine formed
from 100 mM β-AlaOMe and 100 mM L-His

| Microbial cell | Carnosine |
|---|---|
| Rhodotorula minuta IFO0879 | 17.6 |
| Rhodotorula minuta IFO0932 | 19.3 |
| Tremella encephala IFO9293 | 4.46 |
| Rhodotorula minuta Y-33-4 IFO0387 | 5.29 |
| Candida mogii I.G.C.3442 IFO0436 | 14.7 |
| Cryptococcus flavus Y-33-8 IFO0710 | 10.5 |
| Rhodotorula minuta K-38 AJ 4873 | 13.3 |
| Rhodotorula minuta KN-35 CBS 5706 | 9.12 |
| Rhodotorula minuta KN-36 CBS 5695 | 5.72 |
| Rhodotorula minuta AY-24 AJ 4957 | 11.7 |
| Rhodotorula minuta AY-30 AJ 4958 | 18.2 |
| Rhodotorula marina NP-2-10 AJ 4965 | 11.7 |
| Rhodotorula aurantiaca IFO0754 | 2.45 |
| Rhodotorula aurantiaca 68-254 AJ 5119 | 6.18 |
| Erythrobasidium hasegawianum IFO1058 | 13.5 |

As a result, 2.45 to 19.3 mM carnosine accumulated in each microbial strain. Among them, the strains of Rhodotorula minuta IFO0879, Rhodotorula minuta IFO0932, Candida mogii IFO0436, Rhodotorula minuta AY-30 AJ4958 and Erythrobasidium hasegawianum IFO1058 produced the most carnosine, 13.5 mM or more carnosine.

### Example 2

### Purification of the Carnosine-Forming Enzyme Derived from Rhodotorula minuta IFO0879 Strain

The carnosine-forming enzyme was purified from a soluble fraction of the Rhodotorula minuta IFO0879 strain as follows. The enzymatic activity was evaluated by measuring the carnosine-forming activity using β-AlaOMe and L-His as substrates under the following conditions.

Reaction conditions: 50 mM β-AlaOMe, 100 mM L-His and 10 μL/100 μL reaction solution in 100 mM borate buffer (pH 9.0) were reacted at 25° C., and the amount of carnosine which formed after 15 minutes was measured.

(1) Preparation of Soluble Fraction

The Rhodotorula minuta IFO0879 strain was cultured in the same manner as in Example 1. The microbial cells were collected from the resulting culture medium by centrifugation, washed with 50 mM Tris-HCl buffer (pH 7.6), and then collected again by the centrifugation. The resulting microbial cells were suspended in 50 mM Tris-HCl buffer (pH 7.6) and disrupted by sonication at 4° C. for 60 minutes. Microbial cell debris was removed by centrifuging (×8000 rpm, 30 minutes) the disrupted suspension, and the resulting supernatant was used as the soluble fraction.

(2) Ammonium Sulfate Fractionation

Ammonium sulfate was added to the above soluble fraction to 30% saturation. This was centrifuged (×8000 rpm, 30 minutes), and the supernatant was collected. Subsequently, ammonium sulfate was added to the obtained supernatant to 70% saturation. This was centrifuged (×8000 rpm, 30 minutes), and a precipitate was collected. The obtained precipitate was suspended in 50 mM Tris-HCl buffer (pH 7.6), and dialyzed against 50 mM Tris-HCl buffer (pH 7.6) at 4° C. overnight.

(3) Anion Exchange Chromatography: Q-Sepharose FF

The above post-dialysis solution was applied onto an anion exchange chromatography column Q-Sepharose FF 26/10 (supplied from Pharmacia (GE Health Care Bioscience, CV=53 mL), and equilibrated with 50 mM Tris-HCl buffer (pH 7.6) to adsorb proteins to the carrier. The proteins which had not been adsorbed to the carrier (non-adsorbed proteins) were washed out with 50 mM Tris-HCl buffer (pH 7.6). Subsequently, the adsorbed protein was eluted at a flow rate of 8 mL/min with a linearly changing NaCl concentration from 0 M to 0.5 M. The carnosine-forming activity was checked in each eluted fraction, and a peak of a carnosine activity was detected in the fraction corresponding to about 0.3 M NaCl.

(4) Hydrophobic Chromatography: Phenyl Sepharose HP 16/10

The solution in which the carnosine-forming activity had been detected was dialyzed against 1.0 M ammonium sulfate, 50 mM Tris-HCl buffer (pH 7.6) at 4° C. overnight. The resulting solution was applied onto a hydrophobic chromatography column Phenyl Sepharose HP 16/10 (supplied from Pharmacia (GE Health Care Bioscience, CV=20 mL), and equilibrated with 1.0 M ammonium sulfate and 50 mM Tris-HCl buffer (pH 7.6). As a result, the carnosine-forming enzyme was adsorbed to the carrier.

The non-adsorbed proteins that had not adsorbed to the carrier were washed out with 1.0 M ammonium sulfate and 50 mM Tris-HCl buffer (pH 7.6). Subsequently, the carnosine-forming enzyme was eluted at a flow rate of 3 mL/min with a linearly changing ammonium sulfate concentration from 1.0 M to 0 M. The carnosine-forming activity was measured in each eluted fraction, and the carnosine-forming activity was observed in elution positions corresponding to about 0.6 to 0.7 M of the ammonium sulfate concentration.

(5) Gel Filtration Chromatography: SEPHADEX 200 pg 16/60

The fractions containing the carnosine-forming enzyme were combined, which was then dialyzed against 50 mM Tris-HCl buffer (pH 7.6). The resulting solution was concentrated using an ultrafiltration membrane Centriprep 10. The resulting concentrated solution was applied onto gel filtration Sephadex 200 pg 16/60 (supplied from Pharmacia (GE Health Care Bioscience, CV=120 mL) equilibrated with 0.1 M NaCl and 50 mM Tris-HCl buffer (pH 7.6), and eluted at a flow rate of 0.5 mL/min. As a result, the carnosine-forming activity was confirmed at a position estimated to be about molecular weight 230 kDa.

(6) Anion Exchange Chromatography: Mono Q HR 5/5

The obtained fraction was applied onto an anion exchange chromatography column Mono Q HR 5/5 (supplied from Pharmacia (GE Health Care Bioscience, CV=1 mL) equilibrated with 50 mM Tris-HCl buffer (pH 7.6). The carnosine-forming enzyme was adsorbed to the carrier by this manipulation. The non-adsorbed proteins were washed out with 50 mM Tris-HCl buffer (pH 7.6). Subsequently, the carnosine-forming enzyme was eluted at a flow rate of 0.5 mL/min with a linearly changing NaCl concentration from 0 M to 0.5 M. The carnosine-forming activity was measured in each eluted fraction, and confirmed in the elution position corresponding to about 0.3 M of the NaCl concentration.

The resulting fraction was subjected to SDS-PAGE, and two bands corresponding to 30 kDa and 12 kDa were mainly observed in the active fraction. An activity profile and a profile of SDS-PAGE band intensity were matched in the both bands. These two bands were cut out from a SDS-PAGE gel as candidates for the carnosine-forming enzyme, and subjected to the amino acid sequence analysis.

The activities, yields and purification degrees in each purification step are summarized in Table 2.

TABLE 2
Summary of partial purification of the carnosine-forming
enzyme derived from Rhodotorula minuta IFO0879

| | Total | Total | Specific | | Purifi- |
|---|---|---|---|---|---|
| | protein | activity | activity | Yield | cation |
| | (mg) | (U) | (U/mg) | (%) | (fold) |
| Crude extract | 1450 | 32.8 | 0.0226 | 100 | 1.00 |
| Ammonium sulfate | 352 | 31.3 | 0.0889 | 95.4 | 3.93 |
| Q-sepharose | 13.5 | 13.5 | 1.01 | 41.2 | 44.5 |
| Phenyl-sepharose | 0.733 | 12.1 | 16.5 | 37.0 | 732 |
| Superdex | 0.305 | 8.50 | 27.7 | 25.8 | 1230 |
| Mono Q | 0.0967 | 3.12 | 32.3 | 9.51 | 1430 |

1 U = an activity of producing 1 μmol carnosine per one minute

### Example 3

### Determination of N-Terminal and Internal Amino Acid Sequences of Carnosine-Forming Enzyme

After subjecting the purified carnosine-forming enzyme fraction to SDS-PAGE, the band corresponding to 12 kDa was cut out, and the N-terminal amino acid sequence corresponding to 27 amino acid residues was determined as shown in the following Table 3 (SEQ ID NO:8). The band corresponding to 30 kDa was also cut out, the SDS-PAGE gel sample was treated with trypsin (pH 8.0, 35° C., 20 hours), and then subjected to reverse phase HPLC to separate the fragmented peptides. The amino acid sequence corresponding to 12 residues in the separated fraction was determined as shown in the following Table 3 (SEQ ID NO:9). A protein exhibiting significant homology was not detected in the internal amino acid sequence of 30 kDa. However, the N-terminal 12 kDa amino acid sequence exhibited 70% homology to the N-terminal sequence of the DmpA β subunit derived from Ochrobactrum anthropi and 67% homology to the N-terminal sequence of the BapA β subunit derived from Pseudomonas sp. MCI3434.

TABLE 3
Determined amino acid sequences

| | amino acid sequence |
|---|---|
| 12 kDa N-terminal | SIIVVIATDAPLIPIQLQRLAKRATVG |
| amino acid sequence | |
| 30 kDa internal amino | SVIKPADLPHHH |
| acid sequence | |

### Example 4

### Cloning of Gene of the Carnosine-Forming Enzyme Derived from the Rhodotorula minuta IFO0879 Strain

(1) Preparation of cDNA

Rhodotorula minuta IFO0879 strain was applied onto the flat medium containing 10 g/L of glucose, 3 g/L of yeast extract, 3 g/L of malt extract, 5 g/L of peptone and 15 g/L of agar, and cultured at 25° C. for 2 days. One loopful of the resulting microbial cells was inoculated in a liquid medium containing 10 g/L of glucose, 3 g/L of yeast extract, 3 g/L of malt extract, and 5 g/L of peptone in a 500 mL Sakaguchi flask, which was then cultured with shaking at 25° C. for 24 hours. After the cultivation, the microbial cells were collected from the culture by centrifugation. Total RNA was prepared from this microbial cell preparation using RNeasy Midi kit (Qiagen). Subsequently, cDNA was prepared from the resulting total RNA using SMART RACE cDNA Amplification Kit (Clontech).

(2) Acquisition of Sequence of β Subunit of Carnosine-Forming Enzyme by 3′-RACE Method

Mix primers described in Table 4 were synthesized based on the determined N-terminal amino acid sequence of the 12 kDa fragment of the carnosine-forming enzyme.

TABLE 4
Mix primers designed and synthesized according
to N-terminal amino acid sequence

| Name | Sequence (5′-3′) |
|---|---|
| RhDmpA12-f | ATYATYGTNGTNATYGCNACNGAYGCNCC |
| RhDmpA12-f2 | GAYGCNCCNYTNATYCCNATYCARYTNCA |

Amplification by PCR was performed with cDNA of Rhodotorula minuta IFO0879 strain as a template using the produced mix primer RhDmpA12-f (SEQ ID NO:10) and SMART RACE cDNA Amplification Kit (Clontech) Likewise, the amplification by nested PCR using the mix primer RhDmpA12-f2 (SEQ ID NO:11) was performed with the resulting DNA fragment as the template. The obtained DNA fragment was cloned into pTA2 (Takara), and its nucleotide sequence was determined. As a result, the amino acid sequence deduced from the acquired DNA fragment exhibited 44% homology to the amino acid sequence of DmpA β subunit derived from Ochrobactrum anthropi and 41% homology to the amino acid sequence of BapA β subunit derived from Pseudomonas sp. MCI3434. Thus, it was believed that the sequence of the β subunit of the carnosine-forming enzyme had been acquired.

(3) Acquisition of the α Subunit Sequence of the Carnosine-Forming Enzyme by 5′-RACE Method

Primers for 5′-RACE described in Table 5 were formed based on the nucleotide sequence of the β subunit of the carnosine-forming enzyme, which had been determined by the 3′-RACE method.

TABLE 5
Primers designed and synthesized according to
the nucleotide sequence of β-subunit

| Name | Sequence (5′-3′) |
|---|---|
| RhDmpA12-r | CATCAGGGCCTTTTGTATCAGTAGCCATGC |
| RhDmpA12-r2 | TGCGGCTGAGGCACAGAAGGGGTCCAATTC |

The amplification by PCR was performed with cDNA of the Rhodotorula minuta IFO0879 strain as the template using the produced mix primer RhDmpA12-r (SEQ ID NO:12) and SMART RACE cDNA Amplification Kit (Clontech) Likewise, amplification by nested PCR using the mix primer RhDmpA12-f2 (SEQ ID NO:13) was performed with the resulting DNA fragment as the template. The obtained DNA fragment was cloned into pTA2 (Takara), and its nucleotide sequence was determined. As a result, the amino acid sequence deduced from the acquired DNA fragment was homologous to the α subunits of DmpA derived from Ochrobactrum anthropi and BapA derived from Pseudomonas sp. MCI3434. However, the translation initiation site sequence was not confirmed. Thus, primers for 5′-RACE described in Table 6 were synthesized based on the obtained nucleotide sequence.

TABLE 6
Primers designed and synthesized according to
the nucleotide sequence of α-subunit

| Name | Sequence (5′-3′) |
|---|---|
| RhDmpA30-r | AGCCCTCGGGACCATCCATGGGACCAGC |
| RhDmpA30-r2 | CCGCCGAAGTTGCTCTGTACTAATGCCG |

Amplification by PCR was performed with cDNA of Rhodotorula minuta IFO0879 strain as the template using the produced mix primer RhDmpA30-r (SEQ ID NO:14) and SMART RACE cDNA Amplification Kit (Clontech) Likewise, amplification by nested PCR using the mix primer RhDmpA30-r2 (SEQ ID NO:15) was performed with the resulting DNA fragment as the template. The obtained DNA fragment was cloned into pTA2 (Takara), and its nucleotide sequence was determined. As a result, the amino acid sequence deduced from the acquired DNA fragment exhibited 41% homology to the amino acid sequence of DmpA α subunit derived from Ochrobactrum anthropi and 36% homology to the amino acid sequence of BapA α subunit derived from Pseudomonas sp. MCI3434. The translation initiation site sequence was also confirmed. This sequence contained a sequence that was identical to the internal amino acid sequence of the 30 kDa fragment. Thus, it was believed that the α subunit sequence of the carnosine-forming enzyme had been acquired.

(4) Acquisition of Full-Length Gene of Carnosine-Forming Enzyme by PCR

The primers described in Table 7 for amplifying the full-length gene of carnosine-forming enzyme were synthesized from the sequences obtained by 5′-RACE and 3′-RACE.

TABLE 7
Primers designed and synthesized for obtaining
the full-length

| Name | Sequence (5′-3′) |
|---|---|
| RhDmpA-Ndef1 | catATGACCCAAGCAAGAATGTCTTCCC |
| RhDmpA-Hindr | aagcttCTAGTACGCGTGCCGTGTTACAATC |

The amplification by PCR was performed with cDNA of Rhodotorula minuta IFO0879 strain as the template using the produced primers RhDmpA-Ndef1 (SEQ ID NO:16) and RhDmpA-Hindr (SEQ ID NO:17). The resulting DNA fragment was cloned into pTA2 (Takara) and its nucleotide sequence was determined. As a result, this DNA fragment was found to have the nucleotide sequence of SEQ ID NO:1. A 1200 by of ORF including the nucleotide sequences corresponding to the determined N-terminal and internal amino acid sequences was confirmed and the full-length gene of the objective carnosine-forming enzyme (RhDmpA) was acquired. The carnosine-forming enzyme (RhDmpA) was thought to be translated as one polypeptide and subsequently cleaved into the α subunit and the β subunit in the same manner as in DmpA derived from Ochrobactrum anthropi and BapA derived from Pseudomonas sp. MCI3434. It is thought that the α subunit is the amino acid sequence from the first methionine to the 274th glycine in the amino acid sequence of SEQ ID NO:3 and the β subunit is the amino acid sequence from the 275th serine to the 400th tyrosine in the amino acid sequence of SEQ ID NO:3. It has been reported that both polypeptides of DmpA derived from Ochrobactrum anthropi and BapA derived from Pseudomonas sp. MCI3434 is cleaved between glycine and serine into the α subunit and β subunit. The present enzyme is consistent with this point.

Ggenomic DNA was also isolated which includes the polypeptide consisting of the nucleotide sequence of SEQ ID NO:1 as the exon region and includes its intron region. The nucleotide sequence of the isolated genomic DNA was analyzed, and was found to have the nucleotide sequence of SEQ ID NO:32.

### Example 5

### Expression of the Carnosine-Forming Enzyme in E. coli

(1) Construction of a Plasmid Expressing the Carnosine-Forming Enzyme Using the pSFN Vector N

A plasmid to which the gene of the carnosine-forming enzyme had been ligated, pTA2 (Takara), was digested with Nde I and Hind III to obtain a DNA fragment containing the gene of the carnosine-forming enzyme. This was ligated to the vector pSFN in the manner described in WO2006/075486 (see pSFN Sm_Aet in Examples, particularly Examples 1, 6 and 12 in WO2006/075486), which had been previously digested with Nde I and Hind III. E. coli JM109 was transformed with this ligation solution, a strain having the objective plasmid was selected from the ampicillin-resistant strains, and this plasmid was designated as pSFN-RhDmpA. This plasmid expresses the carnosine-forming enzyme consisting of the amino acid sequence of SEQ ID NO:3, which was obtained by translating the nucleotides from the ATG starting from the 40th A as a translation initiation codon to the 1239th nucleotide in the nucleotide sequence of SEQ ID NO:1. At that time, it was thought that the α subunit of the carnosine-forming enzyme consists of the amino acid sequence from the first to the 274th residues in the amino acid sequence of SEQ ID NO: 3, and the β subunit consists of the amino acid sequence from the 275th to 400th residue in the amino acid sequence of SEQ ID NO:3.

(2) Expression of Carnosine-Forming Enzyme in E. coli Using pSFN-RhDmpA

The constructed expression plasmid pSFN-RhDmpA was introduced into E. coli JM109 to obtain a transformant. One loopful of the transformant was inoculated into 50 mL of TB medium containing 100 μg/mL of ampicillin, which was then cultured with shaking at 33° C. for 16 hours. After the cultivation, microbial cells were collected from 1 mL of the resulting culture medium, washed, and suspended in 1 mL of 50 mM Tris-HCl buffer (pH 7.6). The carnosine-forming activity was measured using this microbial cell suspension. The carnosine-forming activity was measured using β-AlaOMe and L-His as the substrates under the following conditions.

Reaction conditions: 50 mM β-AlaOMe, 100 mM L-His and 20 μL of microbial cell suspension/200 μL reaction solution in 100 mM borate buffer (pH 9.0) were reacted at 25° C. for 15 minutes, and the amount of carnosine produced was measured with HPLC.

As a result of the measurement, 2.15 U/mL of carnosine-forming activity was detected in the pSFN-RhDmpA-introduced strain, whereas no carnosine-forming activity was detected in pUC18-introduced E. coli (control). This, in conjunction with the construction of the plasmid with high expression of RhDmpA, confirmed that the gene of the objective carnosine-forming enzyme had been cloned.

(3) Expression of Carnosine-Forming Enzyme from Other Translation Initiation Site

The primers described in Table 8 were synthesized for amplifying ORF encoding the polynucleotide starting from the 55th ATG as the translation initiation codon to the 1239th nucleotide (see SEQ ID NO:4) in the nucleotide sequence of SEQ ID NO:1 and for amplifying ORF encoding the polynucleotide starting from the 91st ATG as the translation initiation codon to the 1239th nucleotide (see SEQ ID NO:6) in the nucleotide sequence of SEQ ID NO:1.

TABLE 8
Primers designed and synthesized for amplifying
ORFs starting from another translation
initiation point

| Name | Sequence (5′-3′) |
|---|---|
| RhDmpA-Ndef2 | catATGTCTTCCCAACCTTCCACCTCAAG |
| RhDmpA-Ndef3 | catATGGAACGAAAGCGTATCCGCGAGC |

The amplification by PCR was performed with cDNA of Rhodotorula minuta IFO0879 strain as the template using the produced primer RhDmpA-Ndef2 (SEQ ID NO:18) and RhDmpA-Hindr. The resulting DNA fragment was cloned into pTA2 (Takara), and its nucleotide sequence was determined and confirmed to have the objective nucleotide sequence. The resulting plasmid was digested with Nde I and Hind III, and then ligated to the pSFN vector which had been digested with Nde I and Hind III. E. coli JM109 was transformed with this ligation solution, a strain having the objective plasmid was selected in ampicillin resistant strains, and this plasmid was designated as pSFN-RhDmpA2. This plasmid expresses the carnosine-forming enzyme consisting of the amino acid sequence of SEQ ID NO:5 obtained by translating the polynucleotide starting from the 55th ATG as the translation initiation codon to the 1239th nucleotide in the nucleotide sequence of SEQ ID NO:1. At this time, it was thought that the α subunit of the carnosine-forming enzyme consists of the amino acid sequence from the first to 269th residues in the amino acid sequence of SEQ ID NO:5 and the β subunit consists of the amino acid sequence from the 270th to 395th residues in the amino acid sequence of SEQ ID NO:5. The constructed expression plasmid pSFN-RhDmpA2 was introduced into E. coli J109 to obtain a transformant. One loopful of the transformants was inoculated in 50 mL of TB medium containing 100 μg/mL of ampicillin, which was then cultured with shaking at 33° C. for 16 hours. After the cultivation, microbial cells were collected from 1 mL of the resulting culture medium, washed, and suspended in 1 mL of 50 mM Tris-HCl buffer (pH 7.6). The carnosine-forming activity was measured using this microbial cell suspension, and found to be 2.09 U/mL.

Likewise, the amplification by PCR was performed using RhDmpA-Ndef3 (SEQ ID NO:19) and RhDmpA-Hindr to construct pSFN-RhDmpA3. This plasmid expresses the carnosine-forming enzyme consisting of the amino acid sequence of SEQ ID NO:7 obtained by translating the polynucleotide starting from the 91st ATG as the translation initiation codon to the 1239th nucleotide in the nucleotide sequence of SEQ ID NO:1. At this time, it was thought that the α subunit of the carnosine-forming enzyme consists of the amino acid sequence from the first to 257th residues in the amino acid sequence of SEQ ID NO:7 and the β subunit consists of the amino acid sequence from the 258th to 383rd residues in the amino acid sequence of SEQ ID NO:7. The constructed expression plasmid pSFN-RhDmpA3 was introduced into E. coli JM109 to obtain a transformant. One loopful of the transformants was inoculated in 50 mL of TB medium containing 100 μg/mL of ampicillin, which was then cultured with shaking at 33° C. for 16 hours. After the cultivation, microbial cells were collected from 1 mL of the resulting culture medium, washed, and suspended in 1 mL of 50 mM Tris-HCl buffer (pH 7.6). The carnosine-forming activity was measured using this microbial cell suspension, and found to be 2.58 U/mL.

### Example 6

### Expression of RhDmpA Homolog in E. coli

(1) Construction of a Plasmid Expressing the RhDmpA Homolog Using the pSFN Vector

Amino acid sequences homologous to RhDmpA3 (hereinafter abbreviated as Rh3 if necessary) were searched for. Expression was attempted for each of the following three homologs (a) to (c), all of which have relatively high homology:

(a) BapA derived from Sphingosinicella microcystinivorans Y2 strain (40% homology to the amino acid sequence of RhDmpA; hereinafter abbreviated as Y2 if necessary);

(b) DmpA derived from Pyrococcus horikoshii OT3 strain (35% homology to the amino acid sequence of RhDmpA; hereinafter abbreviated as PH if necessary); and

(c) DmpA derived from Aspergillus oryzae RIB40 strain (49% homology to the amino acid sequence of RhDmpA; hereinafter abbreviated as As if necessary).

Concerning Y2, Sphingosinicella microcystinivorans Y2 strain (JCM 13185) was obtained from RIKEN Bioresource Center, and its genomic DNA was extracted after its cultivation, and used as the template. A DNA fragment including deduced β-peptidylamine dipeptidase gene (Locus tag number: EF043283, GenBank Accession number: EF043283) was amplified by PCR using the primer Y2-NdeI-f (SEQ ID NO:26) and Y2-HindIII-r (SEQ ID NO:27) described in Table 9. The resulting DNA fragment was digested with Nde I and Hind III to obtain a DNA fragment containing an RhDmpA homolog gene. This DNA fragment was ligated to the vector pSFN described in WO2006/075486, which had been digested with Nde I and Hind III. E. coli JM109 was transformed with this ligation solution, a strain having the objective plasmid was selected from ampicillin-resistant strains, and this plasmid was designated as pSFN-Y2-BapA.

Concerning PH, genomic DNA derived from Pyrococcus horikoshii OT3 strain (JCM 9974, RDB5990) was obtained from RIKEN Bioresource Center, and used as the template. A DNA sequence including a D-aminopeptidase gene (Locus tag number: PH0078, GenBank Accession number: NP_{—}142096 and BA000001) was amplified by PCR using the primer PH-NdeI-f (SEQ ID NO: 28) and the primer PH-HindIII-r (SEQ ID NO:29) described in the following Table 9. The resulting DNA fragment was digested with Nde I and Hind III to obtain a DNA fragment containing an RhDmpA homolog enzyme gene. This DNA fragment was ligated to the vector pSFN described in WO2006/075486 which had been digested with Nde I and Hind III. E. coli JM109 was transformed with this ligation solution, a strain having the objective plasmid was selected from ampicillin-resistant strains, and this plasmid was designated as pSFN-PH-DmpA.

Concerning As, BAC clone B043G02 of genomic DNA derived from Aspergillus oryzae RIB40 was obtained from NBRC, and used as the template. A DNA sequence including L-aminopeptidase/D-esterase gene (Locus tag number: A0090138000075; GenBank Accession number: XM_{—}001825534) was amplified by PCR using the primers As-NdeI-f (SEQ ID NO:30) and As-HindIII (SEQ ID NO:31) described in the following Table 9. The resulting DNA fragment was digested with Nde I and Hind III to obtain a DNA fragment containing the gene of the RhDmpA homolog enzyme. This DNA fragment was ligated to the vector pSFN described in WO2006/075486 which had been digested with Nde I and Hind III. E. coli JM109 was transformed with this ligation solution, a strain having the objective plasmid was selected from ampicillin-resistant strains, and this plasmid was designated as pSFN-As-DmpA.

TABLE 9
Primers designed and synthesized for amplifying
genes of RhDmpA homologs

| Name | Sequence (5′-3′) |
|---|---|
| Y2- | ggaattccatATGCACTATCTGAAATTCCCGGCGATCAT |
| NdeI-f | C |
| Y2- | agcccaagctTCACTTCGCGGCAGCGAGCCGCCTGTACT |
| HindIII-r | TC |
| PH- | ggaattccatATGAAAGCCCAAGAGTTAGGGATTAAAAT |
| NdeI-f | TG |
| PH- | agcccaagctTCATTCCTCCAACCTCCCGTATCTCCTCA |
| HindIII-r | TTATC |
| As- | ggaattccatATGCGCGTCCAGCTATCCCCCGAGCAAGT |
| NdeI-f | AC |
| As- | agcccaagcttCTACACCCGTTGGTATTGCCTCATGATC |
| HindIII-r | TCC |

(2) Enzymatic Expression of RhDmpA Homologs in E. coli

The expression plasmid was introduced into E. coli JM109 to obtain a transformant. One loopful of the transformants was inoculated in 50 mL of TB medium containing 100 μg/mL of ampicillin, which was then cultured with shaking at 37° C. for 16 hours. After the cultivation, microbial cells were collected from 1 mL of the resulting culture medium, washed, and suspended in 1 mL of 50 mM Tris-HCl buffer (pH 7.6). The carnosine-forming activity and a carnosine-degrading activity were measured using this microbial cell suspension.

The carnosine-forming activity was measured using β-AlaOMe and L-His as the substrates under the following conditions.

Reaction conditions: 50 mM β-AlaOMe, 100 mM L-His and 20 μL of microbial cell suspension/200 μL reaction solution in 100 mM borate buffer (pH 8.5) were reacted at 25° C. for 15 minutes, and the amount of carnosine which was produced was measured with HPLC.

The carnosine-degrading activity was measured using carnosine as the substrate under the following conditions.

Reaction conditions: 20 mM carnosine and 20 μL of microbial cell suspension/200 μL reaction solution in 100 mM borate buffer (pH 8.5) were reacted at 25° C. for 15 minutes, and the amount of produced histidine was measured with HPLC.

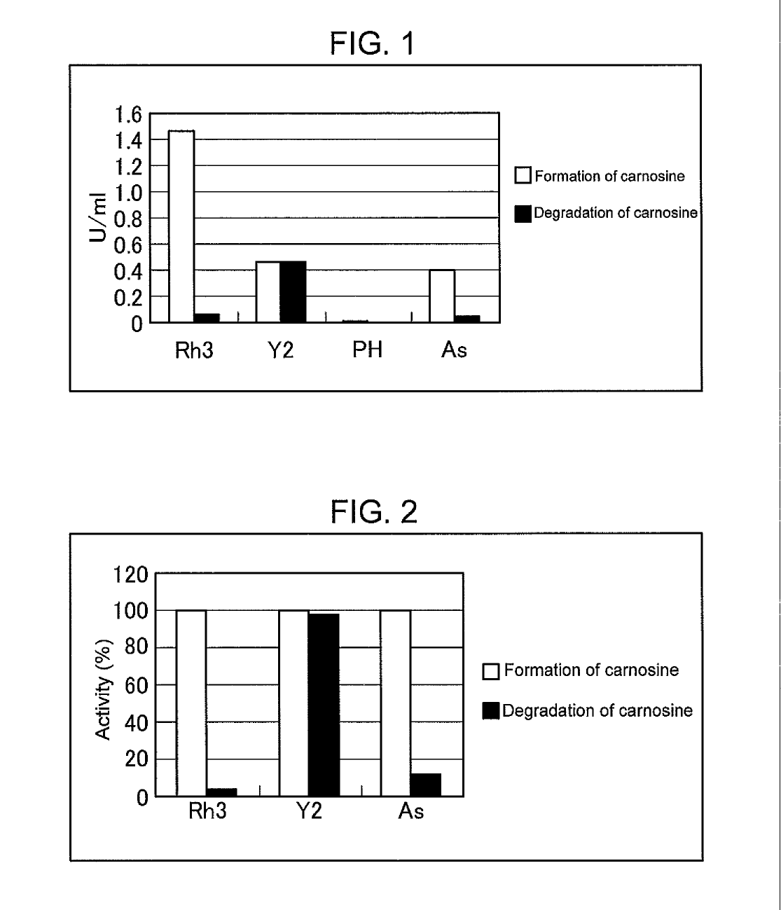

As a result of the measurement, 1.4 U/mL of the carnosine-forming activity was detected in the strain transformed with pSFN-RhDmpA (FIG. 1). About 0.4 U/mL of the activity was also detected in the strain transformed with pSFN-Y2-BapA or pSFN-As-DmpA.

Meanwhile, the carnosine-degrading activity was strong in the strain transformed with pSFN-Y2-BapA (about 0.4 U/mL) while it was faint in the strain transformed with pSFN-Rh DmpA or pSFN-As-DmpA (0.1 U/mL or less). Both the activities were at the detection limit or below in the strain transformed with pSFN-PH-DmpA.

(3) Measurement of Carnosine Yields, and Carnosine Formation and Degradation of RhDmpA Homologs

A carnosine yield was measured for the three strains, i.e., the strains transformed with pSFN-Y2-BapA and pSFN-As-DmpA that were the homologs of pSFN-RhDmpA, the expression of which was identified in (2), and the strain transformed with pSFN-RhDmpA3. As a result, 24%, 31% and 67% yields of carnosine were obtained from the strains transformed with pSFN-Y2-BapA, pSFN-As-DmpA and pSFN-RhDmpA3, respectively. Thus, it was demonstrated that carnosine was efficiently produced (Table 10). From the results of measuring the carnosine formation and degradation, it was thought that not only a specific activity of the carnosine formation but also the ratio of the forming activity and the degrading activity was important (FIG. 2).

TABLE 10
Table 10. Yield of carnosine with each plasmid

| plasmid | yield |
|---|---|
| pSFN-RhDmpA3 | 67% |
| pSFN-Y2-BapA | 24% |
| pSFN-AS-DmpA | 31% |

### Example 7

### Carnosine Formation Reaction Using Substrate Other than Methyl Ester

The RhDmpA enzyme was purified from the strain transformed with pSFN-Rh DmpA3 strain. Using this purified enzyme, carnosine was formed using β-Ala ester or β-Ala amide as the substrate. The carnosine-forming activity was measured using β-Ala ester or β-Ala amide and L-His as the substrates under the following conditions.

Activity measurement reaction conditions: 50 mM β-Ala ester or β-Ala amide, 100 mM L-His and 0.1 U/200 μL reaction solution in 100 mM borate buffer (pH 8.5) were reacted at 25° C. for 15 minutes, and the amount of formed carnosine was measured with HPLC.

Yield measurement reaction condition: 50 mM β-Ala ester or β-Ala amide, 100 mM L-His and 2 U/200 μL reaction solution in 100 mM borate buffer (pH 8.5) were reacted at 25° C. for 120 minutes, and the amount of formed carnosine was measured with HPLC.

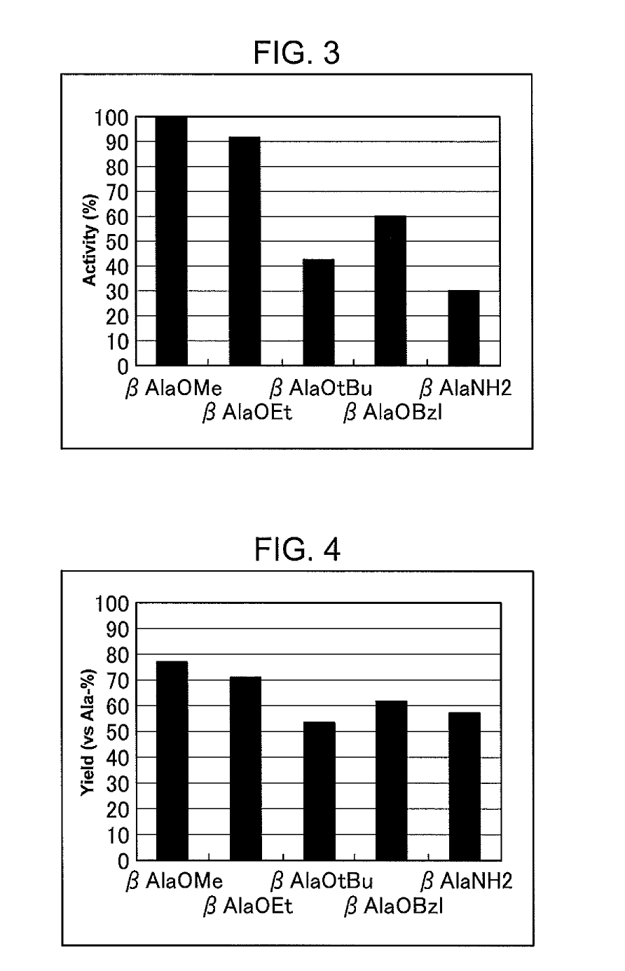

As a result, it was found that esters other than a β-Ala-methyl ester and a β-Ala-amide could be utilized (FIG. 3). The yield was high in the cases of using β-Ala-methyl ester or β-Ala-ethyl ester (FIG. 4).

### Example 8

### β-Ala-X Formation Reaction Using an Amino Acid, X, Other than Histidine as the Substrate

The β-Ala-X formation reaction was performed using the RhDmpA enzyme purified in Example 7, and various amino acids as the substrate. The β-Ala-X-forming activity was measured using β-Ala-methyl ester and the amino acid, X, as the substrates under the following conditions.

Activity measurement reaction conditions: 50 mM β-Ala-methyl ester, 100 mM L-amino acid X, 10 mM EDTA, and 30 mU/200 μL reaction solution in 100 mM borate buffer (pH 9.0) were reacted at 25° C. for 10 minutes, and the amount of produced β-Ala-X was measured with HPLC.

As a result, it was found that the amino acids other than histidine could also be recognized as the substrate.

TABLE 11
Table 11. Specific activities of forming
dipeptides from various amino acids

| amino acid X | produced dipeptide | specific activity (U/mg) |
|---|---|---|
| histidine | carnosine | 28.8 |
| β-alanine | βAla-βAla | 64.8 * |
| alanine | βAla-Ala | 12.5 * |
| valine | βAla-Val | 87.1 |
| phenylalanine | βAla-Phe | 37.5 |

1 U = an activity of forming 1 μmol carnosine per one minute
Only * performed with β-Ala methyl ester at a concentration of 100 mM

### Example 9

### β-Ala-X Formation Reaction Using an Amino Acid Derivative as the Substrate

The β-Ala-X formation reaction was performed using various amino acid derivatives as the substrate in the same manner as in Example 8. The β-Ala-X-forming activity was measured using β-Ala-methyl ester and an amino acid derivative X as the substrate under the following conditions.

Activity measurement reaction condition: 50 mM β-Ala-methyl ester, 100 mM L-amino acid X, 10 mM EDTA, and 30 U/200 μL reaction solution in 100 mM borate buffer (pH 9.0) were reacted at 25° C. for 10 minutes, and the amount of β-Ala-X produced was measured with HPLC.

As a result, it was found that the amino acid derivative could also be recognized as the substrate.

TABLE 12
Table 12. Specific activities of forming dipeptides
from various amino acid derivatives

| derivative X | produced dipeptide | specific activity (U/mg) |
|---|---|---|
| histamine | carcinine | 42.8 * |
| 3-methyl histidine | anserine | 55.4 |
| 1-methyl histidine | vanillin | 12.3 |

1 U = an activity of forming 1 μmol carnosine per one minute
Only * performed with β-Ala methyl ester at a concentration of 100 mM

From the results in Examples 8 and 9, the RhDmpA enzyme is thought to be an enzyme which forms a β-alanyl-amino acid or derivative thereof rather than a carnosine-forming enzyme.

While the invention has been described in detail with reference to preferred embodiments thereof, it will be apparent to one skilled in the art that various changes can be made, and equivalents employed, without departing from the scope of the invention. Each of the aforementioned documents is incorporated by reference herein in its entirety.

1. A method for producing a β-alanyl-amino acid or derivative thereof, said method comprising reacting a substrate selected from the group consisting of a β-alanyl ester and a β-alanyl amide, with an amino acid or a derivative thereof, in the presence of an enzyme or an enzyme-containing product, wherein said enzyme or enzyme-containing product is able to catalyze the formation of a β-alanyl-amino acid or a derivative thereof from said substrate and the amino acid or derivative thereof.

2. The method according to claim 1, wherein said substrate is β-alanyl ester.

3. The method according to claim 1, wherein said amino acid or derivative thereof is an amino acid.

4. The method according to claim 1, wherein said β-alanyl-amino acid or derivative thereof is β-alanyl-histidine, said substrate is β-alanyl ester, and said amino acid or derivative thereof is histidine.

5. The method according to claim 1, wherein said enzyme-containing product is selected from the group consisting of:
A) a culture of a microorganism,
B) a microbial cell separated from a culture of a microorganism,
C) a treated microbial product of a culture of a microorganism, and
D) combinations thereof.

6. The method according to claim 5, wherein said microorganism is of a genus selected from the group consisting of Rhodotorula, Tremella, Candida, Cryptococcus, Erythrobasidium, Sphingosinicella, and Aspergillus.

7. The method according to claim 5, wherein said microorganism is transformed with a gene encoding a protein selected from the group consisting of:
(A) a protein comprising the amino acid sequence of SEQ ID NO:3;
(B) a protein comprising the amino acid sequence of SEQ ID NO:3, but wherein said sequence comprises one or more substitutions, deletions and/or insertions of one or several amino acids, and wherein said protein is able to catalyze the reaction of a substrate selected from the group consisting of a β-alanyl ester and a β-alanyl amide, with an amino acid or a derivative thereof, to form a β-alanyl-amino acid or derivative thereof;
(C) a protein comprising the amino acid sequence of SEQ ID NO:5;
(D) a protein comprising the amino acid sequence of SEQ ID NO:5, but wherein said sequence comprises one or more substitutions, deletions and/or insertions of one or several amino acids, and wherein said protein is able to catalyze the reaction of a substrate selected from the group consisting of a β-alanyl ester and a β-alanyl amide, with an amino acid or a derivative thereof, to form a β-alanyl-amino acid or derivative thereof;
(E) a protein comprising the amino acid sequence of SEQ ID NO:7;
(F) a protein comprising the amino acid sequence of SEQ ID NO:7, but wherein said sequence comprises one or more substitutions, deletions and/or insertions of one or several amino acids, and wherein said protein is able to catalyze the reaction of a substrate selected from the group consisting of a β-alanyl ester and a β-alanyl amide, with an amino acid or a derivative thereof, to form a β-alanyl-amino acid or derivative thereof;
(G) a protein comprising the amino acid sequence of SEQ ID NO:21;
(H) a protein comprising the amino acid sequence of SEQ ID NO:21, but wherein said sequence comprises one or more substitutions, deletions and/or insertions of one or several amino acids, and wherein said protein is able to catalyze the reaction of a substrate selected from the group consisting of a β-alanyl ester and a β-alanyl amide, with an amino acid or a derivative thereof, to form a β-alanyl-amino acid or derivative thereof;
(I) a protein comprising the amino acid sequence of SEQ ID NO:25; and
(J) a protein comprising the amino acid sequence of SEQ ID NO:25, but wherein said sequence comprises one or more substitutions, deletions and/or insertions of one or several amino acids, and wherein said protein is able to catalyze the reaction of a substrate selected from the group consisting of a β-alanyl ester and a β-alanyl amide, with an amino acid or a derivative thereof, to form a β-alanyl-amino acid or derivative thereof.

8. The method according to claim 7, wherein said β-alanyl-amino acid or derivative thereof is β-alanyl-histidine, said substrate is β-alanyl ester, and said amino acid or derivative thereof is histidine.

9. The method according to claim 5, wherein said microorganism is transformed with a polynucleotide selected from the group consisting of:
(a) a polynucleotide comprising the nucleotide sequence of the nucleotide numbers 40 to 1239 of SEQ ID NO:1;
(b) a first polynucleotide which hybridizes under stringent conditions with a second polynucleotide comprising a nucleotide sequence complementary to the nucleotide sequence of nucleotide numbers 40 to 1239 of SEQ ID NO:1, and wherein said first polynucleotide encodes a protein which is able to catalyze the reaction of a substrate selected from the group consisting of a β-alanyl ester and a β-alanyl amide, with an amino acid or a derivative thereof to form a β-alanyl-amino acid or derivative thereof;
(c) a polynucleotide comprising the nucleotide sequence of the nucleotide numbers 55 to 1239 of SEQ ID NO:1;
(d) a first polynucleotide which hybridizes under stringent conditions with a second polynucleotide comprising a nucleotide sequence complementary to the nucleotide sequence of nucleotide numbers 55 to 1239 of SEQ ID NO:1, and wherein said first polynucleotide encodes a protein which is able to catalyze the reaction of a substrate selected from the group consisting of a β-alanyl ester and a β-alanyl amide, with an amino acid or a derivative thereof to form a β-alanyl-amino acid or derivative thereof;
(e) a polynucleotide comprising the nucleotide sequence of the nucleotide numbers 91 to 1239 of SEQ ID NO:1;
(f) a first polynucleotide which hybridizes under stringent conditions with a second polynucleotide comprising a nucleotide sequence complementary to the nucleotide sequence of nucleotide numbers 91 to 1239 of SEQ ID NO:1, and wherein said first polynucleotide encodes a protein which is able to catalyze the reaction of a substrate selected from the group consisting of a β-alanyl ester and a β-alanyl amide, with an amino acid or a derivative thereof to form a β-alanyl-amino acid or derivative thereof;
(g) a polynucleotide comprising the nucleotide sequence of SEQ ID NO:20;
(h) a first polynucleotide which hybridizes under stringent conditions with a second polynucleotide comprising a nucleotide sequence complementary to the nucleotide sequence of SEQ ID NO:20, and wherein said first polynucleotide encodes a protein which is able to catalyze the reaction of a substrate selected from the group consisting of a β-alanyl ester and a β-alanyl amide, with an amino acid or a derivative thereof to form a β-alanyl-amino acid or derivative thereof;
(i) a polynucleotide comprising the nucleotide sequence of SEQ ID NO:24;
(j) a first polynucleotide which hybridizes under stringent conditions with a second polynucleotide comprising a nucleotide sequence complementary to the nucleotide sequence of SEQ ID NO:24, and wherein said first polynucleotide encodes a protein which is able to catalyze the reaction of a substrate selected from the group consisting of a β-alanyl ester and a β-alanyl amide, with an amino acid or a derivative thereof to form a β-alanyl-amino acid or derivative thereof;
(k) a polynucleotide comprising the nucleotide sequence of SEQ ID NO: 32; and
(l) a first polynucleotide which hybridizes under stringent conditions with a second polynucleotide comprising a nucleotide sequence complementary to the nucleotide sequence of SEQ ID NO:32, and wherein said first polynucleotide encodes a protein which is able to catalyze the reaction of a substrate selected from the group consisting of a β-alanyl ester and a β-alanyl amide, with an amino acid or a derivative thereof to form a β-alanyl-amino acid or derivative thereof.

10. The method according to claim 1, wherein said enzyme is selected from the group consisting of:
(A) a protein comprising the amino acid sequence of SEQ ID NO:3;
(B) a protein comprising the amino acid sequence of SEQ ID NO:3, but wherein said sequence comprises one or more substitutions, deletions and/or insertions of one or several amino acids, and wherein said protein is able to catalyze the reaction of a substrate selected from the group consisting of a β-alanyl ester and a β-alanyl amide, with an amino acid or a derivative thereof, to form a β-alanyl-amino acid or derivative thereof;
(C) a protein comprising the amino acid sequence of SEQ ID NO:5;
(D) a protein comprising the amino acid sequence of SEQ ID NO:5, but wherein said sequence comprises one or more substitutions, deletions and/or insertions of one or several amino acids, and wherein said protein is able to catalyze the reaction of a substrate selected from the group consisting of a β-alanyl ester and a β-alanyl amide, with an amino acid or a derivative thereof, to form a β-alanyl-amino acid or derivative thereof;
(E) a protein comprising the amino acid sequence of SEQ ID NO:7;
(F) a protein comprising the amino acid sequence of SEQ ID NO:7, but wherein said sequence comprises one or more substitutions, deletions and/or insertions of one or several amino acids, and wherein said protein is able to catalyze the reaction of a substrate selected from the group consisting of a β-alanyl ester and a β-alanyl amide, with an amino acid or a derivative thereof, to form a β-alanyl-amino acid or derivative thereof;
(G) a protein comprising the amino acid sequence of SEQ ID NO:21;
(H) a protein comprising the amino acid sequence of SEQ ID NO: 21, but wherein said sequence comprises one or more substitutions, deletions and/or insertions of one or several amino acids, and wherein said protein is able to catalyze the reaction of a substrate selected from the group consisting of a β-alanyl ester and a β-alanyl amide, with an amino acid or a derivative thereof, to form a β-alanyl-amino acid or derivative thereof;
(I) a protein comprising the amino acid sequence of SEQ ID NO:25;
(J) a protein comprising the amino acid sequence of SEQ ID NO: 25, but wherein said sequence comprises one or more substitutions, deletions and/or insertions of one or several amino acids, and wherein said protein is able to catalyze the reaction of a substrate selected from the group consisting of a β-alanyl ester and a β-alanyl amide, with an amino acid or a derivative thereof, to form a β-alanyl-amino acid or derivative thereof; and
(K) combinations thereof.

11. A protein derived from a microorganism belonging to a genus selected from the group consisting of Rhodotorula, Tremella, Candida, Cryptococcus, and Erythrobasidium, and wherein said protein is able to catalyze the reaction of a substrate selected from the group consisting of a β-alanyl ester and a β-alanyl amide, with an amino acid or derivative thereof, to form a β-alanyl-amino acid or derivative thereof.

12. A protein selected from the group consisting of:
(A) a protein comprising the amino acid sequence of SEQ ID NO:3;
(B) a protein comprising the amino acid sequence of SEQ ID NO: 3, but wherein said sequence comprises one or more substitutions, deletions and/or insertions of one or several amino acids, and wherein said protein is able to catalyze the reaction of a substrate selected from the group consisting of a β-alanyl ester and a β-alanyl amide, with an amino acid or a derivative thereof, to form a β-alanyl-amino acid or derivative thereof;
(C) a protein comprising the amino acid sequence of SEQ ID NO:5;
(D) a protein comprising the amino acid sequence of SEQ ID NO: 5, but wherein said sequence comprises one or more substitutions, deletions and/or insertions of one or several amino acids, and wherein said protein is able to catalyze the reaction of a substrate selected from the group consisting of a β-alanyl ester and a β-alanyl amide, with an amino acid or a derivative thereof, to form a β-alanyl-amino acid or derivative thereof;
(E) a protein comprising the amino acid sequence of SEQ ID NO:7; and
(F) a protein comprising the amino acid sequence of SEQ ID NO: 7, but wherein said sequence comprises one or more substitutions, deletions and/or insertions of one or several amino acids, and wherein said protein is able to catalyze the reaction of a substrate selected from the group consisting of a β-alanyl ester and a β-alanyl amide, with an amino acid or a derivative thereof, to form a β-alanyl-amino acid or derivative thereof.

13. The protein according to claim 12, wherein said β-alanyl-amino acid or derivative thereof is β-alanyl-histidine, said substrate is β-alanyl ester, and said amino acid or derivative thereof is histidine.

14. A polynucleotide encoding the protein according to claim 12.

15. A polynucleotide selected from the group consisting of:
(a) a polynucleotide comprising the nucleotide sequence of nucleotide numbers 40 to 1239 of SEQ ID NO:1;
(b) a first polynucleotide which hybridizes under stringent conditions with a second polynucleotide comprising a nucleotide sequence complementary to the nucleotide sequence of nucleotide numbers 40 to 1239 of SEQ ID NO:1, and wherein said first polynucleotide encodes a protein which is able to catalyze the reaction of a substrate selected from the group consisting of a β-alanyl ester and a β-alanyl amide, with an amino acid or a derivative thereof, to form a β-alanyl-amino acid or derivative thereof;
(c) a polynucleotide comprising the nucleotide sequence of nucleotide numbers 55 to 1239 of SEQ ID NO:1;
(d) a first polynucleotide which hybridizes under stringent conditions with a second polynucleotide comprising a nucleotide sequence complementary to the nucleotide sequence of nucleotide numbers 55 to 1239 of SEQ ID NO:1, and wherein said first polynucleotide encodes a protein which is able to catalyze the reaction of a substrate selected from the group consisting of a β-alanyl ester and a β-alanyl amide, with an amino acid or a derivative thereof, to form a β-alanyl-amino acid or derivative thereof;
(e) a polynucleotide comprising the nucleotide sequence of nucleotide numbers 91 to 1239 of SEQ ID NO:1;
(f) a first polynucleotide which hybridizes under stringent conditions with a second polynucleotide comprising a nucleotide sequence complementary to the nucleotide sequence of the nucleotide numbers 91 to 1239 of SEQ ID NO:1, and wherein said first polynucleotide encodes a protein which is able to catalyze the reaction of a substrate selected from the group consisting of a β-alanyl ester and a β-alanyl amide, with an amino acid or a derivative thereof, to form a β-alanyl-amino acid or derivative thereof;
(g) a polynucleotide comprising the nucleotide sequence of SEQ ID NO:32; and
(h) a first polynucleotide which hybridizes under stringent conditions with a second polynucleotide comprising a nucleotide sequence complementary to the nucleotide sequence of SEQ ID NO:32, and wherein said first polynucleotide encodes a protein which is able to catalyze the reaction of a substrate selected from the group consisting of a β-alanyl ester and a β-alanyl amide, with an amino acid or a derivative thereof, to form a β-alanyl-amino acid or derivative thereof.

16. The polynucleotide according to claim 15, wherein said stringent conditions comprise washing at a salt concentration corresponding to 1×SSC and 0.1% SDS at 60° C.

17. A recombinant polynucleotide comprising the polynucleotide according to claim 14.

18. A recombinant polynucleotide comprising the polynucleotide according to claim 15.

19. A recombinant polynucleotide comprising the polynucleotide according to claim 16.

20. A cell transformed with the polynucleotide according to claim 17.

21. A cell transformed with the polynucleotide according to claim 18.

22. A cell transformed with the polynucleotide according to claim 19.


SEQUENCE LISTING (32 sequences)

SEQ ID NO: 1 - DNA, 1293 bp, Rhodotorula minuta
attcgtccattctttctcctcctcccctgcccgccttcaatgacccaagcaagaatgtcttcccaaccttccacctcaagcagcagtagcatggaacgaaagcgtatccgcgagcttctgccgaaccttcgttttggacaatttccaacaggacctaagaacagcttgacggatgttcccggtgttctagtgagcacgaagagcgtcatcaagcctgcggacttgccgcatcatcatgaagtgaatacgggtgtcactacgattctgccgcgcaaagaatggttcaagcaaggctgctatgcttcgtattttcgcttcaatggatccggagagatgaccggcagtcattggattgacgaaagtggactgctgaactcgcctgtcatcatcacaaactcgttcggcgtgggcgcttgctacaatggcgtatatgaatacgcaaagaaacaccacaaagacgaaaaagggatatgcgactggtttttgactcctgtcattgctgaaactttcgatggatggctcagcgatatcggtgcgatggctgttcagagctctgatgttgtcgaaggcattgagaacgcttcaagtgatgctgtaccggaaggttgcacgggaggaggtactggaatgatcacaatgggcttcaaggcaggaacaggcaacgcaagccgtgtcatcgacagcgtcaagatcgattcaaaaggcgaaaagcagcaagtcaagtatacgctagcggcattagtacagagcaacttcggcggagcacgctttttgactgtcaatggtgtgccagtcgggcgcatattggaggatgaagcgatggccgctaagaaagctggtcccatggatggtcccgagggctcgattatcgtcgtcatagctacagatgcacctctcattccaattcaattgcagcgactggcgaaaagagcgactgttggcgtggcaagaactggaggatggggaagcaactactctggcgatatcttccttgcattttcaacagctcatgagattccacgagaaaatacgcagaattggaccccttctgtgcctcagccgcaggaagtactggatacagaaagtataaatgcattattcgaagctgcttttgaagctgtcgaggaggctatctacaatgctatttgcatggctactgatacaaaaggccctgatggaagggaggtcaaggctatagatctggaaaagctgaaagagattgtaacacggcacgcgtactagagtggggctcaagacgccttaggcattccgcatacaaagactactgctacg

SEQ ID NO: 2 - DNA, 1293 bp, Rhodotorula minuta; identical to SEQ ID NO: 1
  CDS (40)..(1239): gene coding carnosine-forming enzyme

SEQ ID NO: 3 - protein, 400 aa, Rhodotorula minuta
MTQARMSSQPSTSSSSSMERKRIRELLPNLRFGQFPTGPKNSLTDVPGVLVSTKSVIKPADLPHHHEVNTGVTTILPRKEWFKQGCYASYFRFNGSGEMTGSHWIDESGLLNSPVIITNSFGVGACYNGVYEYAKKHHKDEKGICDWFLTPVIAETFDGWLSDIGAMAVQSSDVVEGIENASSDAVPEGCTGGGTGMITMGFKAGTGNASRVIDSVKIDSKGEKQQVKYTLAALVQSNFGGARFLTVNGVPVGRILEDEAMAAKKAGPMDGPEGSIIVVIATDAPLIPIQLQRLAKRATVGVARTGGWGSNYSGDIFLAFSTAHEIPRENTQNWTPSVPQPQEVLDTESINALFEAAFEAVEEAIYNAICMATDTKGPDGREVKAIDLEKLKEIVTRHAY

SEQ ID NO: 4 - DNA, 1293 bp, Rhodotorula minuta; identical to SEQ ID NO: 1
  CDS (55)..(1239): gene coding carnosine-forming enzyme

SEQ ID NO: 5 - protein, 395 aa, Rhodotorula minuta
MSSQPSTSSSSSMERKRIRELLPNLRFGQFPTGPKNSLTDVPGVLVSTKSVIKPADLPHHHEVNTGVTTILPRKEWFKQGCYASYFRFNGSGEMTGSHWIDESGLLNSPVIITNSFGVGACYNGVYEYAKKHHKDEKGICDWFLTPVIAETFDGWLSDIGAMAVQSSDVVEGIENASSDAVPEGCTGGGTGMITMGFKAGTGNASRVIDSVKIDSKGEKQQVKYTLAALVQSNFGGARFLTVNGVPVGRILEDEAMAAKKAGPMDGPEGSIIVVIATDAPLIPIQLQRLAKRATVGVARTGGWGSNYSGDIFLAFSTAHEIPRENTQNWTPSVPQPQEVLDTESINALFEAAFEAVEEAIYNAICMATDTKGPDGREVKAIDLEKLKEIVTRHAY

SEQ ID NO: 6 - DNA, 1293 bp, Rhodotorula minuta; identical to SEQ ID NO: 1
  CDS (91)..(1239): gene coding carnosine-forming enzyme

SEQ ID NO: 7 - protein, 383 aa, Rhodotorula minuta
MERKRIRELLPNLRFGQFPTGPKNSLTDVPGVLVSTKSVIKPADLPHHHEVNTGVTTILPRKEWFKQGCYASYFRFNGSGEMTGSHWIDESGLLNSPVIITNSFGVGACYNGVYEYAKKHHKDEKGICDWFLTPVIAETFDGWLSDIGAMAVQSSDVVEGIENASSDAVPEGCTGGGTGMITMGFKAGTGNASRVIDSVKIDSKGEKQQVKYTLAALVQSNFGGARFLTVNGVPVGRILEDEAMAAKKAGPMDGPEGSIIVVIATDAPLIPIQLQRLAKRATVGVARTGGWGSNYSGDIFLAFSTAHEIPRENTQNWTPSVPQPQEVLDTESINALFEAAFEAVEEAIYNAICMATDTKGPDGREVKAIDLEKLKEIVTRHAY

SEQ ID NO: 8 - protein, 27 aa, Rhodotorula minuta
SIIVVIATDAPLIPIQLQRLAKRATVG

SEQ ID NO: 9 - protein, 12 aa, Rhodotorula minuta
SVIKPADLPHHH

SEQ ID NO: 10 - DNA, 29 bp, Artificial
  primer RhDmpA12-f
atyatygtngtnatygcnacngaygcncc

SEQ ID NO: 11 - DNA, 29 bp, Artificial
  primer RhDmpA12-f2
gaygcnccnytnatyccnatycarytnca

SEQ ID NO: 12 - DNA, 30 bp, Artificial
  primer RhDmpA12-r
catcagggccttttgtatcagtagccatgc

SEQ ID NO: 13 - DNA, 30 bp, Artificial
  primer RhDmpA12-r2
tgcggctgaggcacagaaggggtccaattc

SEQ ID NO: 14 - DNA, 28 bp, Artificial
  primer RhDmpA30-r
agccctcgggaccatccatgggaccagc

SEQ ID NO: 15 - DNA, 28 bp, Artificial
  primer RhDmpA30-r2
ccgccgaagttgctctgtactaatgccg

SEQ ID NO: 16 - DNA, 28 bp, Artificial
  primer RhDmpA-Ndef1
catatgacccaagcaagaatgtcttccc

SEQ ID NO: 17 - DNA, 31 bp, Artificial
  primer RhDmpA-Hindr
aagcttctagtacgcgtgccgtgttacaatc

SEQ ID NO: 18 - DNA, 29 bp, Artificial
  primer RhDmpA-Ndef2
catatgtcttcccaaccttccacctcaag

SEQ ID NO: 19 - DNA, 28 bp, Artificial
  primer RhDmpA-Ndef3
catatggaacgaaagcgtatccgcgagc

SEQ ID NO: 20 - DNA, 1191 bp, Sphingosinicella microcystinivorans Y2
  CDS (1)..(1191)
atgcactatctgaaattcccggcgatcatcgcgggcatgcttctggcgggcgcggcgagtgcggaggggccgcgcgcgcgggatctcggcgtgccgtttgccgggaagccgggcgcgaacaatgcgatcaccgatgttgcgggggtcgaggtcggttacgtcagcctgatttcgggcgagggcaagctggaacgcggcaaggggccggtgcgcacgggcgtgaccgccgtgctgccgcgcggcaaggagtctcgcacgcccgtgtacgcgggctgggaaaccagcaacgccgccggggagatgaccggcacggtgtggctggaggagcgcggctatttcgacgggccgatgatgatcaccaacacgcacagcgtcggcgtggtgcgcgatgcggtggtggggtggcttgcggacgtgaaatggccgggggcgtggttcacgccggtggtggccgaaacctacgacggtatgctgaacgacatcaacggcttccacgtgaagcccgagcacgcgctgcgcgcgatccagaccgcggcatccggcccggtcgccgagggcaatgtcggcggcggcgtcgggatgcagtgcttcggcttcaagggcggcacgggcacggcctcgcgcgtggtcgaaatggatggcaagagctacacggtcggcgtgctcgtgcagtgcaacttcggcatgcggccgtggctgcgcgtggcgggtgcgccggtgggcgaggaactcgcgggcaagtacctccccgaaacacgcgggacgcaaacggcggcggcgacgaataacggcgtcgcgccgggagacggctcaatcatcgtggtgatggcaacggatgcaccgatgctgccgcatcagctgaagcgcctcgccaagcgcgcggcggcgggcatgggccgcatgggcgacgcgggcagcaacggctcaggcgacatcttcgtcgctttctcgaccgcgaacgcaaatgtgcagagcgtgggcggcaatgtcatcagcgtcgagacgatgcccaacgacaagctgacgctgatcttcgaagcggcgacgcaggcgaccgaagaggcgatcaccaacgtgctggtggcagcggacacgctgaccggtgtcaacggctacaccatccagcgcctgccgcacgcggaactgcgggcgattttgaagaagtacaggcggctcgctgccgcgaagtga

SEQ ID NO: 21 - protein, 396 aa, Sphingosinicella microcystinivorans Y2
MHYLKFPAIIAGMLLAGAASAEGPRARDLGVPFAGKPGANNAITDVAGVEVGYVSLISGEGKLERGKGPVRTGVTAVLPRGKESRTPVYAGWETSNAAGEMTGTVWLEERGYFDGPMMITNTHSVGVVRDAVVGWLADVKWPGAWFTPVVAETYDGMLNDINGFHVKPEHALRAIQTAASGPVAEGNVGGGVGMQCFGFKGGTGTASRVVEMDGKSYTVGVLVQCNFGMRPWLRVAGAPVGEELAGKYLPETRGTQTAAATNNGVAPGDGSIIVVMATDAPMLPHQLKRLAKRAAAGMGRMGDAGSNGSGDIFVAFSTANANVQSVGGNVISVETMPNDKLTLIFEAATQATEEAITNVLVAADTLTGVNGYTIQRLPHAELRAILKKYRRLAAAK

SEQ ID NO: 22 - DNA, 1086 bp, Pyrococcus horikoshii OT3
  CDS (1)..(1086)
atgaaagcccaagagttagggattaaaattggcgtgtttaagccagggaagagaaacaaaataactgacgttaaaggagttaaagtgggacacgtaacattaatcaagggaaaagggaagctgataccaggaaagggaccagtaagaacaggtgttaccgcaatactaccacacgaagggaacatatacaaagagaaagttctagcaggagctttcgtaatgaacgggtattctaagccagttggcttgatccagctttgggagctgggaactatagaaaccccaataatattgacgaacacgttgagcattggaacggccgttgaaggccttttagattacattctagaggagaatgaagacataggcgttacaactggttcagttaaccccctagtgctggagtgcaacgattcataccttaatgacatcaggggaaggcacgtcaagagggaacatgtggttgaggcaataaagagagcagatgaagactttgaagaaggagcagtaggagctggaaccggaatgagcgcctttgaattcaaaggaggaataggatccgcttcaaggatagtggaaatagagggtaagaaatataccgtgggggcactagttcttagcaactttggaaggagggaagatctaacgatcgctggagttccagttggattggaacttaaaaattggccaggaagagggggagagggaaaagggagcattataatgataatagcaaccgatgcccccctaacgggtaggcaattaaatagggtagccaagagggccattgtagggctggccagaacgggtggatacgcttacaatgggagcggggatatagcggtagcattctcaacggccaataggattaaacactatgaaaaggaggtcatcgaaataaaagcgctcccagattctgtaatctctcccctgtttaaggccactgctgaagcagtggaagaggccataataaattccctgctagaggcaagaacgatggatggaagagataaccatgttaggtatgcacttccaaaagaggagttgctaaggataatgaggagatacgggaggttggaggaatga

SEQ ID NO: 23 - protein, 361 aa, Pyrococcus horikoshii OT3
MKAQELGIKIGVFKPGKRNKITDVKGVKVGHVTLIKGKGKLIPGKGPVRTGVTAILPHEGNIYKEKVLAGAFVMNGYSKPVGLIQLWELGTIETPIILTNTLSIGTAVEGLLDYILEENEDIGVTTGSVNPLVLECNDSYLNDIRGRHVKREHVVEAIKRADEDFEEGAVGAGTGMSAFEFKGGIGSASRIVEIEGKKYTVGALVLSNFGRREDLTIAGVPVGLELKNWPGRGGEGKGSIIMIIATDAPLTGRQLNRVAKRAIVGLARTGGYAYNGSGDIAVAFSTANRIKHYEKEVIEIKALPDSVISPLFKATAEAVEEAIINSLLEARTMDGRDNHVRYALPKEELLRIMRRYGRLEE

SEQ ID NO: 24 - DNA, 1161 bp, Aspergillus oryzae RIB40
  CDS (1)..(1161)
atgcgcgtccagctatcccccgagcaagtaccgcggcgaatgcgcatccgcgaacttcttcccgacctcgacctgggagcctaccccccaggtccgctaaattccatcaccgacgtgcctggcgtgcacgttcacacccaagagatattcggtgcccaaggcgccatcaataccggcgtgacctgcatcgttccccgcccgaactggtccaccaatgcttgttacgccggggtcttccgttttaacggctcgggagagctaacgggcgcacatctgatagaagagacggggcttctctgctcccctattgttctcactggaacatttaatattggggccgcccaccaagggatctatcagtatgccgtcaaacacctggggactaacaaggatggtcagttggagtggcttatgttgccggtcgtgggtgagacgttcgatgggtatttgcatgactgtacatcgttcgctgtggctcctgcgcacatcgtgcacggtctggagagtgtggtggctggggagccagttcgagagggtaatgtgggcggtggcgtgggcatggtttgccatggactgaaagggggtaccgggagcagcagtcgccaggttctcggaacctacacggtcgcagcgttggtccaggctaattacgggcaattaagagatctccgcattgcaggggtccctgttgggaaaatactaactgaagacgctgcgagcgacccctcaagacaggggatgtatgaggaggtcgcacaggcaaaggccgagaaggacggcagtataatcgttgtactggccacagatgcgccgcttcatcccgctcagttgcagcgtgttgccaagcgggcaaccgttggtcttgctcgcgttggtggccaaggacataacctgtccggggatatcttcctagctttctctactgggaacgagatacctgtcaaccagcacaagagacctgcgtcagtagcacgaactattgacgtgctggatgattctgctctcaataccttgttcgaagcgaccgccgatgcggttgaagaggcgatatacaacgcactttgtatggcggagagccttcaaggttttcaagggcatacaatcgaggcacttcctcttgcccgtctgaaggagatcatgaggcaataccaacgggtgtag

SEQ ID NO: 25 - protein, 386 aa, Aspergillus oryzae RIB40
MRVQLSPEQVPRRMRIRELLPDLDLGAYPPGPLNSITDVPGVHVHTQEIFGAQGAINTGVTCIVPRPNWSTNACYAGVFRFNGSGELTGAHLIEETGLLCSPIVLTGTFNIGAAHQGIYQYAVKHLGTNKDGQLEWLMLPVVGETFDGYLHDCTSFAVAPAHIVHGLESVVAGEPVREGNVGGGVGMVCHGLKGGTGSSSRQVLGTYTVAALVQANYGQLRDLRIAGVPVGKILTEDAASDPSRQGMYEEVAQAKAEKDGSIIVVLATDAPLHPAQLQRVAKRATVGLARVGGQGHNLSGDIFLAFSTGNEIPVNQHKRPASVARTIDVLDDSALNTLFEATADAVEEAIYNALCMAESLQGFQGHTIEALPLARLKEIMRQYQRV

SEQ ID NO: 26 - DNA, 40 bp, Artificial
  primer Y2-NdeI-f
ggaattccatatgcactatctgaaattcccggcgatcatc

SEQ ID NO: 27 - DNA, 41 bp, Artificial
  primer Y2-HindIII-r
agcccaagcttcacttcgcggcagcgagccgcctgtacttc

SEQ ID NO: 28 - DNA, 41 bp, Artificial
  primer PH-NdeI-f
ggaattccatatgaaagcccaagagttagggattaaaattg

SEQ ID NO: 29 - DNA, 44 bp, Artificial
  primer PH-HindIII-r
agcccaagcttcattcctccaacctcccgtatctcctcattatc

SEQ ID NO: 30 - DNA, 41 bp, Artificial
  primer As-NdeI-f
ggaattccatatgcgcgtccagctatcccccgagcaagtac

SEQ ID NO: 31 - DNA, 42 bp, Artificial
  primer As-HindIII-r
agcccaagcttctacacccgttggtattgcctcatgatctcc

SEQ ID NO: 32 - DNA, 1420 bp, Rhodotorula minuta
  exon (1)..(575)
  exon (650)..(987)
  exon (1045)..(1227)
  exon (1314)..(1420)
atgacccaagcaagaatgtcttcccaaccttccacctcaagcagcagtagcatggaacgaaagcgtatccgcgagcttctgccgaaccttcgttttggacaatttccaacaggacctaagaacagcttgacggatgttcccggtgttctagtgagcacgaagagcgtcatcaagcctgcggacttgccgcatcatcatgaagtgaatacgggtgtcactacgattctgccgcgcaaagaatggttcaagcaaggctgctatgcttcgtattttcgcttcaatggatccggagagatgaccggcagtcattggattgacgaaagtggactgctgaactcgcctgtcatcatcacaaactcgttcggcgtgggcgcttgctacaatggcgtatatgaatacgcaaagaaacaccacaaagacgaaaaagggatatgcgactggtttttgactcctgtcattgctgaaactttcgatggatggctcagcgatatcggtgcgatggctgttcagagctctgatgttgtcgaaggcattgagaacgcttcaagtgatgctgtaccggaaggttgcacggggtgagacgacaatgcattacagtgaggactggtgtctcagatccatccaagctgacgattgattctgttgacagaggaggtactggaatgatcacaatgggcttcaaggcaggaacaggcaacgcaagccgtgtcatcgacagcgtcaagatcgattcaaaaggcgaaaagcagcaagtcaagtatacgctagcggcattagtacagagcaacttcggcggagcacgctttttgactgtcaatggtgtgccagtcgggcgcatattggaggatgaagcgatggccgctaagaaagctggtcccatggatggtcccgagggctcgattatcgtcgtcatagctacagatgcacctctcattccaattcaattgcagcgactggcgaaaagagcgactgttggcgtggcaagaagtaagtactgtcttcgagtatcagcgatattccgcagagcctaacgcatttccccagctggaggatggggaagcaactactctggcgatatcttccttgcattttcaacagctcatgagattccacgagaaaatacgcagaattggaccccttctgtgcctcagccgcaggaagtactggatacagaaagtataaatgcattattcgaagctgcttttgaagctgtcgaggaggctatctgtaagtctgtactaattgcagcctttctccagtaagtaatgacagcgcagctgctgatgattgcactccttcttatatgacgatagacaatgctatttgcatggctactgatacaaaaggccctgatggaagggaggtcaaggctatagatctggaaaagctgaaagagattgtaacacggcacgcgtactag